(19) United States
(12) Patent Application Publication
(10) Pub. No.: US 2004/0152868 A1
(43) Pub. Date: Aug. 5, 2004

(54) Compositions and methods for modulating guanylyl cyclase signaling receptor (gc-c) activity and for treating meniere's disease

(76) Inventors: Bjarne Due Larsen, Bronshoj (DK); Soren Neve, Lyngby (DK); Klaus Ovortrup, Virum (DK); Eddi Meier, Vaerlose (DK); Soberg J. Peterson, Gentogte (DK)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/473,841

(22) PCT Filed: Mar. 29, 2002
(86) PCT No.: PCT/IB02/02466

(30) Foreign Application Priority Data
Mar. 30, 2001 (DK) PA200100534

(51) Int. Cl. C07K 7/64; A61K 38/12

(52) U.S. Cl. 530/317; 514/9

(57) ABSTRACT

Disclosed are compositions and methods for modulating guanylyl cyclase signaling receptor (GC-C). Further disclosed are highly useful in vitro and in vivo screens for detecting compounds that modulate the GC-C receptor. The invention has a wide spectrum of useful applications including providing therapeutic compounds that can be employed to prevent and/or treat inner ear disorders and particularly Ménière's disease.

### CROSS-REFERENCE TO RELATED APPLICATION

The present application claims priority to Danish application no. PA200100534 filed on 30 Mar. 2001 the disclosure of which is incorporated herein by reference.

### FIELD OF THE INVENTION

The present invention generally relates to compositions and methods for modulating guanylyl cyclase signaling receptor (GC-C). Preferred compounds are useful for the prevention or treatment of inner ear disorders and particularly Ménière's disease. Further included are pharmaceutical compositions and use of same for treatment of Ménière's disease. Also provided are useful screens for detecting compounds that modulate the GC-C receptor and novel guanylin analogues and a method of preparing said analogues.

### BACKGROUND

Disorders affecting the inner ear have attracted much scientific investigation. For example, Ménière's disease is a disorder described in 1861 by Prosper Ménière. It is characterised by a constellation of symptoms referred to as Ménière's triad and includes fluctuating hearing level, roaring ringing in the affected ear (tinnitus), and episodic vertigo. There is usually a low tone hearing loss, which fluctuates unpredictably. A sensation of fullness in the ear or a sensation that the ear is clogged is also not uncommon. The roaring tinnitus is usually of a low tone quality typically described as “ocean waves crashing along the beach”. Vertigo is a sensation of surrounding objects spinning around you or that you are physically being spun around. The duration of an attack is from minutes to hours. The frequency of these attacks can vary from once a year to three times a day. The symptoms begin suddenly often without prodromes. Patients are completely incapacitated during an attack.

For a review, see generally Qvotrup K. et al. In Ménière's disease—Perspectives in the 90's (Filipo R. et al. eds. ) Kugler Publications, Amsterdam, The Netherlands (1994) 427.

Histopathologically, Ménière's disease is characterised by an increased amount of fluid in the endolymphatic compartment of the inner ear. The inner ear (cochlea and vestibular apparatus is filled with two types of fluids called the endolymph and the perilymph. They differ in their sodium and potassium content. This difference is believed to be responsible for the DC potential (+90 mV) of the endolymph. Two types of epithelial cells in the inner ear presumably control the regulation/production/maintenance of endolymph: The vascular stria in the cochlea and the vestibular dark cells in the vestibular apparatus.

The endolymph is formed/maintained within the cochlea and vestibular apparatus. An extension of the inner ear, known as the endolymphatic duct and sac drains the endolymph. The endolymphatic saclies against the dura in close relation to the sigmoid sinus. Within the endolymphatic sac, endolymph is filtered through to the connective tissue around the sac. From here the fluid can enter blood vessels or enter the spinal fluid space. The volume of endolymph in the inner ear is extremely small (2-3 microL).

Endolymphatic hydrops (excessive fluid within the endolymphatic space) may be caused by a number of diseases that can affect the rest of the body. These include metabolic disorders such as diabetes, low or over-active thyroid functioning, high cholesterol or triglyceride levels. Finally, there are a number of autoimmune and infectious disorders which can also cause these symptoms.

Morphological investigations of the endolymphatic sac (saccus endolymphaticus) revealed that it consisted of two distinct types of cells: The mitochondrial rich cells and the chief cells. The great number of Golgi apparatus complexes consisting of 5-8 cisterns characterize the chief cells. Numerous small electron dense vesicles have been observed in the vicinity of the Golgi complexes suggesting that these vesicles are involved in membrane trafficking from the Golgi apparatus to the lumen of the endolymphatic sac. The endolymphatic sac has been reported to produce a natriuretic hormone. See Qvortrup, K. et al. (1996) Am. Journal. Physiol. 270 (Renal Fluid Electrolyte Physiol. 39) F 1073.

Currently, the initial line of treatment for episodic vertigo is modification of the patient's diet. Since it is thought that excessive fluid within the inner ear leads to many of the symptoms, attempts at reducing salt intake can help in reducing the water content of the inner ear. Additionally, caffeine and nicotine can be stimulants to the vestibular system and cause episodes of vertigo. If episodes of vertigo persist despite changes in diet, antihistamines such as Betaserc have also proven effective in the symptomatic treatment of vertigo in Ménière's disease. Vastarel is an antiischemic drug which has also proven effective in alleviating symptoms of Ménière's disease diuretics are frequently administrated.

Another line of treatment of Ménière's disease involves the injection of the antibiotic, Gentamicin, through the eardrum into the middle ear. The antibiotic is then taken up by the inner ear. Gentamicin is toxic to the hair cells of the hearing and balance organs. The idea is to destroy enough of the hair cells to eliminate the vertigo. There are advantages and disadvantages of this form of treatment. The attractive feature is that surgery is not required. One of the major drawbacks of this treatment is the risk of hearing loss. When gentamicin reaches the middle ear, absorption into the inner ear is uncontrolled and variable. Multiple injections may be required in order to achieve the desired effect.

Another option is a surgical procedure to help drain the endolymphatic sac. Of the surgical options, this is the most conservative operation with minimal risk to hearing. Unfortunately, control of vertigo occurs in only 50-60 percent of patients undergoing this operation.

Vestibular neurectomy is an operation to divide the balance nerves and interrupt the connection between the inner ear and the brain. 90-95 percent of patients who undergo this operation for Ménière's disease will not have another spell of vertigo. After the operation, dysequilibrium lasts until the brain learns to compensate and adapt to the lack of input from one inner ear.

Labyrinthectomy is an operation to surgically remove a portion of the inner ear. Nearly all patients with Ménière's disease will be vertigo-free after a labyrinthectomy. Total and complete hearing loss will result in the operated ear. This is an operation that is recommended to patients that receive no useful hearing in the dysfunctional ear. Just as in vestibular neurectomy, after the operation dysequilibrium lasts until the brain learns to compensate and adapt to the lack of input from one inner ear.

There have been attempts to understand second messenger systems. One important system is that activated by membrane bound guanylyl cyclase signalling receptor (sometimes called herein guanylyl cyclase C or GC-C). GC-C has been described as a member of the family of membrane-bound guanylyl cyclases that synthesises cGMP. That molecule is believed to function as a second messenger in intestinal and kidney cortex epithelial cells in response to stimulation by a ligand.

There is acknowledgement that GC-C receptor activation is via the endogenous peptide hormone, guanylin. There is understanding that GC-C greatly increases cGMP levels in intestinal epithelial cells on interaction with an exogenous peptide. A heat-stable enterotoxin produced by enteric bacteria such as Eschedichia coil is also thought to assist activation of the cyclase.

More specifically, GC-C has been disclosed as being a single subunit protein molecule (1050 residues) with a unique structure consisting of an N-terminal extracellular domain, which is responsible for ligand binding and a C-terminal intracellular domain that is responsible for catalysis of cGMP synthesis. The two domains are thought to be connected by a single transmembrane polypeptide. The N-terminal domain is specific for the ligands, such as guanylin. There is recognition that upon binding of ligand, binding information is transferred to the C-terminal domain via the transmembrane polypeptide. Binding results in the stimulation of the guanylyl cyclase catalytic region in the intracellular domain. The molecular mechanisms for ligand recognition and the signal transduction by GC-C are currently unknown, but one plausible scenario is that GC-C induces a change in conformation or topology between the domains when ligand binding occurs, resulting in the removal of a negative regulatory effect and thus creating a favorable environment for the synthesis of cGMP.

There have been attempts to identify GC-C in the inner ear. See Hearing Res, 1997, 110:95-106 (describing presence of GC-C mRNA in the inner ear tissues, cochlea and vestibular organ in guinea pig). Attempts to detect presence of membrane-bound guanylyl cyclase has not been successful in the inner ear.

Guanylin has been reported to be an intestinal peptide hormone of 15 amino acids that activates the guanylinyl cyclase C receptor. It contains two disulfide bonds with 1-3 and 24 cysteine connectivity, which is crucial for the biological activity. Unlike other multiple disulfide-bridged peptides, spectroscopical investigations have demonstrated that guanylin contains two structurally distinct species that differ in the backbone conformation while containing the same disulfide connectivity. The two forms are called topoisomers. The two isomers exist in a dynamic equilibrium and interconvert without disulfide opening and rearrangement. It was found impossible to isolate the topoisomers of guanylin due to the rapid interconversion.

There have been reports that the GC-C receptor is activated by E. coli heat-stable enterotoxin (Sta).

The guanylin peptide has been grouped into a family that includes uroguanylin and lymphoguanylin. The peptide has been reported in the intestinal tract, adrenal gland and kidney. In the kidney, guanylin has been disclosed as exerting a diuretic, natriuretic and kaliuretic effect. See Currie, M. G. et al. (1992) PNAS (USA); and Fonteles, M. C (1998) Am. J. Physiol. 275: F191-F197.

It would be useful to have compounds that modulate the GC-C receptor. It would be particularly useful to have compounds that help maintain fluid homeostasis in the inner ear and prevent or treat Ménière's disease. It would also be useful to have methods for making and detecting such compounds.

### SUMMARY OF THE INVENTION

The present invention generally relates to compositions and methods for modulating the guanylate cyclase C receptor (GC-C). In one embodiment, the invention can be used to treat Ménière's disease including symptoms of the disease such as vertigo, hearing loss, and tinnitus. Further provided are methods for making such compounds. The invention has a broad spectrum of uses including providing in vitro and in vivo screens for detecting compounds that modulate GC-C activity.

We have found that the endolymphatic sac of the inner ear (saccus endolymphaticus) functions as a new endocrine tissue or gland. The active sac factor is often referred to herein as saccin. As discussed above, the factor has been shown to induce natriuresis. We have also found that the inner ear expresses guanylin and the GC-C receptor. Further, we have learned that saccin and guanylin show essentially the same diuretic activity. Without wishing to be bound to theory, we believe that guanylin and saccin assist inner ear homeostasis, particularly by maintaining fluid electrolyte balance.

More specifically, it has been shown that saccin produces a significant diuretic, natriuretic and kaliuretic effect. See (Qvortrup, K., et al. Am J Physiol, 270, F1073-F1077 (1996)). Blood pressure, glomerular filtration and lithium clearance effects have been shown to be minimal. Guanylin, like saccin, has also been shown to have a substantial diuretic, natriuretic and kaliuretic effect. In addition, saccin produces a significant increase in cGMP formation corresponding to the effect of guanylin. This and other information indicates to us that saccin and guanylin are essentially the same peptide. This and other information further indicates that guanylin is a potent endocrine hormone that associates with GC-C and helps maintain electrolyte balance in fluids of the inner ear. Without wishing to be bound to theory, it is believed that unsuitable guanylin levels interact unfavorably with the GC-C receptor to disrupt, normal fluid electrolyte balance. This disruption is further believed to facilitate onset of or aggravate symptoms of Ménière's disease.

Accordingly, and in one embodiment, the invention provides compositions and methods that modulate the GC-C receptor, particularly by increasing or decreasing association binding between guanylin ligand and the receptor. By “modulate” is meant increase or decrease. Particular compositions of the invention include guanylin and biologically active fragments thereof; guanylin analogues; and cyclic peptides. Preferred compositions have capacity to bind guanylin, the GC-C receptor or both. Also included within the scope of this invention are particular GC-C peptide fragments that correspond to those parts of the GC-C receptor sequence found in or near the receptor binding site. Particular fragments of interest associate with guanylin and preferably bind thereto to help reduce or eliminate ligand binding to the receptor. Specific examples of such compositions as well as novel methods for making same are disclosed below.

A particular invention object is to provide such compounds, compositions and methods for the treatment of Ménière's disease. More specific compositions and methods decrease catalysis of cGMP by GC-C preferably by inhibiting association between guanylin and the GC-C receptor.

Hence, it is one objective of the present invention to provide a compound capable of inhibiting ligand binding to guanylyl cyclase C and/or capable of inhibiting synthesis of cGMP (via stimulation of GC-C). In one invention embodiment, the compound comprises a biologically active fragment of guanylin; a guanylin analogue; or a cyclic polypeptide of the invention such as those capable of inhibiting guanylyl cyclase activity of the GC-C receptor.

It is furthermore an objective of the present invention to provide an antibody that specifically binds to a guanylyl cyclase C polypeptide which comprises an epitope comprising the amino acid sequence Pro-Xaa-Phe-Xaa-Trp, and wherein said antibody is capable of binding said epitope.

It is yet another objective of the present invention to provide an isolated monoclonal antibody that specifically binds to a guanylyl cyclase C epitope and wherein said epitope preferably comprise the amino acid sequence Xaa-Pro-Xaa-Phe-Xaa-Trp.

A further objective of the present invention is to provide a pharmaceutical composition comprising a therapeutically effective amount of at least one of the compounds disclosed herein, preferably an inhibitor, wherein said inhibitor is capable of inhibiting binding of guanylin to the GC-C receptor. Additionally preferred inhibitors reduce or eliminate GC-C activity. Illustrative inhibitors according to the invention include biologically active fragments of guanylin; guanylin analogues, cyclic polypeptides, biologically active fragments of the GC-C receptor and antibodies that specifically binds to the GC-C receptor.

Furthermore, it is an objective of the present invention to provide a use of said pharmaceutical composition together with a pharmaceutically acceptable carrier for the preparation of a medicament.

It is also an objective of the present invention to provide a use of said pharmaceutical composition together with a pharmaceutically acceptable carrier for the preparation of a medicament for treatment of one or more symptoms of Ménière's disease.

Yet another objective of the present invention is to provide a method of treatment of one or more symptoms of Ménière's disease, comprising administrating to an individual in need thereof the pharmaceutical composition comprising a therapeutically effective amount of an inhibitor, wherein said inhibitor is capable of inhibiting binding of Guanylin or a compound which comprises guanylin like activity to Guanylyl Cyclase C.

In preferred aspects the present invention provides compositions comprising soluble GC-C polypeptides, antibodies that are specifically immunoreactive with GC-C polypeptides, and methods of using these compositions in screening, therapeutic and diagnostic applications.

There has been recognition that guanylin and related compounds are often difficult to make particularly when standard solid phase methodologies are employed. Typically, a crude material containing only about 20% of the target peptide is the unwanted result. Linear primary peptide intermediates that are made during the synthesis are often insoluble in water. Subsequent purification of the compounds using standard routes can be almost impossible. These and other problems have made producing and using guanylin and related compounds difficult and costly.

The invention addresses these problems by providing easier and more cost effective methods for making guanylin and related compounds, such as uroguanylin, E. coli heat-stable enterotoxin (Sta) and lymphoguanylin. The methods are broadly applicable and can be used to help synthesis of other “problem” peptides. As will be appreciated, such peptides typically have at least two cysteine residues therein eg., between from about four to about ten of such residues. Presence of such residues in relatively small peptides can make them difficult to make and use when standard techniques are used.

Accordingly, the invention provides methods for making such “problem” peptides that generally involves adding at least one charged amino acid to the peptide preferably under conditions that favor reversible (or hydrolyzable) bonding between the C-terminus of the peptide and the charged amino acid. Such a peptide is sometimes referred to herein as being tailed or “SIPed”. It has been found that by modifying the peptide in accord with the intention it is possible to provide for more effective peptide synthesis. It has also been found that such SIPed peptides are more stable. In a preferred embodiment, the addition of the charged amino acid(s) is(are) reversible. That is, addition is achieved to controllably remove/separate the amino acid(s) from the peptide. Illustrative conditions to assist the removal or separation include, but are not limited to, chemical, photochemical and/or enzymatic cleavage of the amino acid(s) from the tailed peptide. The method can be used to make a wide range of peptides such as mammalian guanylin including biologically active fragments and analogues thereof.

The invention further relates to methods for detecting and analyzing compounds that modulate the GC-C receptor and preferably exhibit therapeutic capacity to treat or prevent diseases associated with abnormal receptor function including inner ear disorders involving imbalance of fluid electrolytes such as Ménière's disease. Preferred detection and analysis methods include in vitro and in vivo assays to determine capacity of candidate compounds to increase or decrease receptor activity.

Accordingly, and in one aspect, the invention provides a method for screening compounds for capacity to modulate a membrane bound guanylyl cyclase signaling receptor (GC-C). In one embodiment, the assay involves at least one and preferably all of the following steps: a) contacting cells expressing the GC-C with at least one compound under conditions sufficient to bind a control (eg., guanylin ligand in vehicle) to the receptor; and b) detecting the binding of the compound in the presence of the ligand in which an increase or decrease in binding is taken to be indicative of the compound that modulates the GC-C receptor.

In another embodiment, the in vitro screening method includes at least one and preferably all of the following steps: a) contacting cells expressing the GC-C with the compound under conditions conducive to forming cGMP; and b) measuring the cGMP produced by the cells where a change in cGMP production relative to a control (vehicle) is taken to be indicative of the compound that modulates the GC-C. Such an assay will sometimes be referred to herein as a cGMP-efficacy assay.

The in vitro assays can be used, individually or together, to detect and optionally measure capacity of one or more of the compounds to increase or decrease GC-C activity relative to a control (eg., guanylin). Reference herein to a “standard in vitro GC-C assay” or related phrase, refers to combined use of both of the above in vitro protocols. Such a combined assay format is very flexible and can be employed with nearly any population of cells that include the GC-C receptor. These cells include, but not limited to, a membrane containing lysate of such cells or tissue. Suitable whole cells for use with the assay include primary, secondary, or immortalized cells.

The invention further provides highly useful in vivo assays that can be used to determine therapeutic capacity of a known or candidate GC-C receptor in a test animal. In general, the assays detect and optionally measure impact of the compounds on one or more of diuresis, natriuresis, kaliuresis, hearing, or heart function in the animal. As discussed below, such in vivo assays can be used alone, in combination with each other, or in combination with one or more of the in vitro assays disclosed herein.

In one embodiment, the monitored condition in the test animal includes detecting and measuring diuresis, preferably also natriuresis, more preferably natriuresis and kaliuresis along with the diuersis measurement. Such a method will generally include screening compounds for capacity to modulate the GC-C receptor in which at least one of the following steps is practiced:

a) administering a candidate compound to the animal under conditions suitable for detecting diuresis, preferably associated with a sustained natriuretic response that outlasts the diuretic response, and b) measuring the diuresis and preferably also urinary sodium excretion produced by the animal, wherein presence of diuresis relative to a control (vehicle) is taken to be indicative of capacity to modulate the GC-C in the animal. Reference herein to a “standard diuresis assay” refers to the above protocol of steps a) and b). Such an assay is flexible and can be used to measure urine output and preferably also sodium and/or potassium ion content by standard methods and with nearly any type of mammal such as rodents (eg, rats and mice), rabbits, as well as primates including monkeys, chimpanzees and the like.

In another embodiment, the invention provides another in vivo assay that suitably detects and optionally measures changes in animal hearing following administration of the test compound. In a preferred embodiment, the monitored hearing function is otoacoustic emission after between from about a few days to about a few weeks or more following administration of the test compound.

As an illustration, the invention provides methods for screening compounds for capacity to modulate the GC-C receptor in the test animal that includes at least one and preferably all of the following steps: a) administering a GC-C receptor agonist to the animal under conditions suitable for detecting otoacoustic emission; b) administering a candidate compound exhibiting GC-C receptor antagonist or inhibitor activity to the animal under conditions suitable for detecting otoacoustic emission; and c) measuring the otoacoustic emission. Preferably, the emission when relatively of the same magnitude relative to a control (vehicle) is taken to be indicative of capacity to antagonise the effect of the GC-C agonist in the animal. Reference herein to a “standard otoacoustic assay” or related phrase refers to the above protocol of steps a)-c). Such an assay is flexible and can be used to detect and preferably measure changes in hearing function in nearly any type of mammal such as rodents (eg, rats and mice), rabbits, as well as primates including monkeys, chimpanzees and the like.

If desired, one or more of the foregoing in vitro and/or in vivo tests can be combined with a standard test for measuring heart function eg., arterial pressure, heart beats per minute and the like. Alternatively, such heart assays can be used as the sole screening method although in many embodiments, pre-screening one or more candidate compounds in accord with one or more of the in vitro methods of the invention will be highly desirable.

Accordingly, and in one embodiment, the invention further includes in vivo assays in which impact of one or more of the candidate compounds is measured on the heart of a suitable test animal such as those already mentioned. Preferably, the candidate compound is administered to the subject and then heart function, preferably arterial pressure and/or cardiac beats per minute is (are) evaluated. Preferred invention compounds suitably increase arterial pressure in the test animal by at least about 5% to about 50% when compared to a suitable control such as vehicle. Typical compounds will exhibit such effects within about an hour, preferably between from about 5 minutes to about 45 minutes. Additionally preferred compounds will feature no or a negligible heart rate changes compared to the control.

Such broad spectrum testing provided by the present invention provides advantages. Thus, for example, the in vitro assays provided herein can be used to perform multiple and/or repeated assays, thereby enhancing efficiency and probability of identifying useful GC-C modulating compounds. This feature of the invention is especially useful when large compound populations are to be handled. For instance, libraries of candidate peptide compounds can be made by standard peptide/combinatorial type chemical manipulations and then tested. Significantly, use of multiple detection platforms (eg., a combination of in vitro and in vivo assays) with one or more candidate compounds can extend desired selectivity and sensitivity of the invention.

In another aspect, the invention features use of the compounds disclosed herein to prevent or treat fluid and sodium retention (eg., associated with heart failure, liver cirrhosis and nephrotic syndrome) and hypotension. Thus in one embodiment there is provided a method for facilitating diuresis in a mammal in which the method includes administering a therapeutically effective amount of at least one of: guanylin or a biologically active fragment thereof; a guanylin analogue or a cyclic peptide to the mammal.

The invention further provides antibodies that bind to the GC-C receptor. In one embodiment, there is provided use of a peptide sequence of or an epitopic fragment of the GC-C receptor preferably coupled to a carrier through a terminal cysteinyl residue for raising antibodies capable of specifically binding to said peptide.

Particularly provided is use of Compound 11 H-SPNFITKC—NH₂ or Compound 11A H-SPNFIWKC—NH₂ for raising an antibody capable of specifically binding to a GC-C receptor or a fraction of the peptide sequence thereof.

It is believed that guanylin peptides and certain analogues thereof possess antiproliferative activity in intestinal cells. Accordingly, it is an object of the invention to use the compounds disclosed herein to prevent or treat colorectal cancer. Preferred compounds will reduce or eliminate colonic carcinoma.

### BRIEF DESCRIPTION OF THE DRAWINGS

FIG. 1 is a graph showing stimulation of cGMP formation in T84 cells grown to 70% confluence in 48-multi well culture plates. A two site fit algorithm was used.

FIG. 2 is a photomicrograph showing localization of guanylin in saccus endolymphaticus.

FIG. 3 is a diagram showing results of guanylin receptor tracing.

FIG. 4 is a graph showing changes in arterial pressure following administration of guanylin analogue.

FIG. 5 is a graph showing changes in heart rate (beats per minute) following administration of guanylin analogue.

FIG. 6 is a graph showing effect of guanylin analogue on urine flow rate.

FIG. 7 is a graph showing effect of guanylin analogue on urinary sodium excretion rate.

FIG. 8 is a graph showing effect of guanylin analogue on urinary potassium excretion rate.

FIG. 9 is a graph showing sodium excretion facilitated by saccus endolymphaticus crude extract.

FIG. 10 is a graph showing change in otoacoustic emission after 2 weeks i.p. treatment with vehicle or Compound 1 relative to pre-treatment level.

FIG. 11 is a graph showing a DP-gram 1 week after administration of Compound 1 to a rat.

FIG. 12 is a graph showing an active DP-gram following administration of Compound 1 to left and right ears of a rat.

FIG. 13 is a graph showing a DP-gram delta following administration of Compound 1 to a rat.

FIG. 14 is a graph showing showing stimulation by Compound 1 of cGMP formation in T84 cells.

FIG. 15 is a graph showing stimulation of cGMP formation by saccin-extract in T84 cells.

FIG. 16 is a graph showing stimulation of cGMP formation in T84 cells by Compound 11.

### DETAILED DESCRIPTION OF THE INVENTION

As discussed, the present invention relates to compounds capable of inhibiting ligand binding to the guanylyl cyclase C receptor (GC-C). Additionally preferred compounds are capable of inhibiting synthesis of cGMP through stimulation of GC-C. It is believed that Ménière's disease is associated with a hyperproduction of guanylin in the endolymphatic sac, and that guanylin via the GC-C receptor in the inner ear vascular stria causes a reduced resorption of sodium from the endolymph. This change in sodium ion concentration in the endolymph is thought to assist hydrops formation and facilite onset of symptoms indicative of the disease.

Thus the present invention relates, in one aspect, to compounds that competitively inhibit guanylin or guanylin-like compounds from binding to GC-C. These compounds may have one or more desirable activities eg., binding to the GC-C receptors or to guanylin or the guanylin-like compounds. The compounds may be selected from compounds such as polypeptides, peptides, oligomers, antibodies, small molecules, small organic molecules as well as combinations thereof. Preferred compounds include biologically active fragments of guanylin; guanylin analogues; cyclic peptides having capacity to associate with guanylin and/or the GC-C receptor; as well as specific GC-C fragments that bind guanylin and reduce or eliminate further ligand binding to the receptor.

Suitable guanylins according to the invention are preferably mammalian guanylins, more preferably rat, mouse, human, pig, opossum or guinea pig guanylin. By the phrase “biologically active fragment” of guanylin or guanylin analogue is meant a portion of a guanylin or analogue thereof that exhibits at least about 50% of the activity of a control mammalian guanylin (eg., rat or human guanylin), more preferably at least about 70% of that activity in the standard in vitro GC-receptor assay. Examples of such fragments include guanylin analogues in which the first or first and second N-terminal amino acids have been deleted.

As discussed, the standard in vitro GC-C assay preferably involves determining: 1) GC-C receptor binding and 2) cGMP production.

A preferred GC-C receptor binding assay includes the following steps: a) culturing suitable cells in medium such as T84 cells and adding about 1 micromolar to about 5 micromolar of one or more detectably-labeled candidate compounds (eg., iodinated or tritiated) or vehicle in the presence of about 10 micromolar unlabeled ligand (guanylin). Preferably, the culturing is conducted under conditions conducive to binding the compound and ligand to the GC-C receptor; and b) detecting binding of the detectably-labeled compound to the receptor. Preferred invention compounds will have an IC50 of less than about 100 μM, preferably less than about 50 μM. Still further preferred invention compounds provide an IC50 of about 1 μM or less, preferably between about 1 μM and about 50 μM in the same assay. See Example 1 below.

Typically, that in vitro assay is combined with a cGMP production or “efficacy” assay that involves the following steps: a) culturing suitable cells in medium eg., T84 cells and adding about 1 micromolar to about 25 micromolar of one or more of the candidate compounds, b) lysing the cells after about 5 minutes to about 1 hour; and c) determining the amount of cGMP in the lysate in the presence and absence of the test compound. See Example 1 below.

Determination of whether a compound is capable of stimulating catalysis of cGMP by GC-C can be done by any method known to the person skilled in the art. Preferably, such method is the method outlined in example 1 herein. Sometimes that method will be referred to herein as an “efficacy assay”. Specifically preferred compounds have an EC50 of less than about 100 μM, preferably less than about 50 μM. Still further preferred invention compounds provide an EC50 of about 1 μM or less, preferably between about 1 μM and about 50 μM in the same assay. See Example 1 below.

In embodiments in which GC-C enhancers are to be screened according to the standard in vitro GC-C detection assay, typically preferred compounds will exhibit at least about 20%, preferably at least about 50%, more preferably at least about 100% to about 250% of the activity of a suitable control such as a mammalian guanylin in vehicle.

As discussed, additionally preferred invention compounds e.g, the biologically active guanylin fragments, guanylin analogues and cyclic peptides disclosed herein, exhibit significant diuretic activity ie., they increase rate of urine formation in a mammal. As used herein, “diuresis” means an increase in urine volume and net loss of solute and water. More particular compounds enhance sodium ion loss (natriuretic) in the urine. In many embodiments, such compounds will also manifest detectable increases in potassium ion loss (kaliuretic) in the urine. Diuretic compositions and methods for monitoring diuresis are known and have been disclosed. See generally Goodman L. S. and Gilman, A. in The Pharmacological Basis of Therapeutics (5^{th} Ed.) MacMillan Publishing Co. Inc. NY (1975).

The standard diuresis assay of the invention is flexible and can be readily adaptable to suit an intended screening application. Preferred compounds detected by the in vivo assay will feature an increase in urine output of at least about 20%, preferably at least about 100%, more preferably at least about 150% to about 200% when compared to administration of a suitable control such as vehicle. More preferred invention compounds will exhibit a sustained natriuretic response (ie. enhanced output of urine sodium ion) of at least about 10 minutes post-administration of the compound, preferably at least about 60 minutes, more preferably at least about 150 minutes. Still more preferred compounds will feature an increase in kaliuresis soon after administration of the compound (ie., within about 1 to about 10 minutes or more). Preferably, the increase will be at least about 20% higher, more preferably at least about 50% higher, and even more preferably at least about 100% to about 150% higher when compared to a suitable control such as vehicle.

As an example, the standard diuresis assay can be practiced using rats. Typically, a catheter or related device is inserted into the urinary bladder to facilitate collection of urine. For injection of compounds, an i.v. line into a major artery will be preferred. After a recovery period, the rats are given a liquid such as glucose water. If desired, heart function can be monitored according to standard procedures in this assay. In most cases, a bolus injection of the test compound will be preferred. Subsequently, urine is collected using conventional procedures. In embodiments in which sodium and/or potassium ion amounts are desired, the ions can be analyzed using one or a combination of more routine methods eg., a flame spectrometer or related device. See Example 4 below.

There have been no reports of an in vivo model of Ménière's disease. This drawback has hindered attempts to develop effective therapies. The present invention addresses this need by providing a highly useful rat model of the disease. The model has a wide spectrum of uses including providing a system for screening compounds that modulate the GC-C receptor and preferably also reduce or eliminate symptoms associated with Ménière's disease. Accordingly, it is an object of the present invention to provide additionally preferred compounds that are capable of modulating hearing in the rat model including compounds that are capable of inhibiting or antagonising the effect of guanylin as a GC-C receptor agonist.

Of particular interest are compounds that reduce hearing in a rat model of experimental hydrops when compared to a suitable vehicle or carrier in the standard otoacoustic emission test. Examples of said compounds are guanylin and guanylin analogues as described herein. Measurements of hearing loss are not particularly related to a specific frequency range, but it is preferred to be able to measure otoacoustic emission in the rat hearing range up to about 70 kHz and it is preferred that the measurable hearing loss is of the same order of magnitude in both ears in relation to a control group. More preferred compounds in accord with the invention provide a change in otoacoustic emission of at least about 5 dB preferably at least about 7.5 dB, more preferably about 10 dB or more in the test. See the discussion and examples below. See Example 5, below.

Methods for measuring otoacoustic emission in mammals such as rodents are known. Particular methods are disclosed in the examples and discussion that follows. Preferred invention compounds will exert a demonstrable effect on hearing after a few days and more preferably about one to about two to three weeks after administration of the compound.

The present in vitro and in vivo assays can be used to screen a wide spectrum of mammalian guanylins, guanylin analogues, guanylin or guanylin analogue fragments with biological activity. Such assays can also be used to screen known or suspected inhibitors of GC-C (membrane associated guanylate cyclase receptor). Examples of such compounds include guanylyl cyclase inhibiting protein and low molecular weight compounds, such as methylene blue; Zinc protoporphyrin IX; 1H[1,2,4]oxadiazolo[4,3-a]quinoxalin-1-one (ODQ); 6-(phenylamino)-5,8-quinolinedione (LY 83.583), which inhibits the catalytic domain of both soluble and particulate (membrane bound) guanylyl cyclases (Kook et al.; Eur J Pharmacol 1999 Jan 22;365(2-3):267-72); and the active site cyclase inhibitors guanosine 5′-tetraphosphate and adenosine 5′-tetraphosphate and the cyclase inhibitor Rp-GTPalphaS (Garger et al.; Vis Neurosci 2001 Jul-Aug;18(4):625-32). These compounds are all useful in the treatment of diseases related to hyperstimulation of GC-C receptor, e.g. as a result of hypersecretion of guanylin. Particular disease states are Ménière's disease or one or more of the symptoms associated with Ménière's disease such as tinnitus, vertigo and hearing loss. Inhibitors of GC-C in the intestine may also be useful in the treatment of diarrhea and especially diarrhea caused by heat stable enterotoxin (Sta) produced by pathogenic Escherichia coli in the intestine.

Suitable GC-C receptor agonists include guanylin and analogues thereof, eg., mammalian uroguanylin, E. coli heat-stable enterotoxin (Sta). Additional in vivo methods for analyzing guanylin activity have been disclosed. See Carrithers, et al. (1999) Brazilian J. of Med. And Biol. Res. 32: 1337.

It is one objective of the present invention to provide antibodies that are specifically immunoreactive with Guanylyl Cyclase C. The phrase “specifically immunoreactive,” when referring to the interaction between an antibody of the invention and a particular protein, refers to an antibody that specifically recognises and binds with relatively high affinity to the protein of interest, e.g., Guanylyl Cyclase C. Thus, under designated immunoassay conditions, the specified antibodies bind to a particular protein and do not bind in a significant amount to other proteins present in the sample. A variety of immunoassay formats may be used to select antibodies specifically immunoreactive with a particular protein. For example, solid-phase ELISA immunoassays are routinely used to select monoclonal antibodies specifically immunoreactive with a protein. See, Harlow and Lane (1988) Antibodies, A Laboratory Manual, Cold Spring Harbor Publications, New York, for a description of immunoassay formats and conditions that can be used to determine specific immunoreactivity.

In one aspect of the present invention the antibodies are polyclonal antibodies. For production of polyclonal antibodies, an appropriate target immune system is selected, typically a mouse or rabbit, but also including goats, sheep, cows, guinea pigs, monkeys, donkeys and rats. The substantially purified antigen is presented to the immune system in a fashion determined by methods appropriate for the animal. These and other parameters are well known to immunologists. Typically, injections are given in the footpads, intramuscularly, intradermally or intraperitoneally. The immunoglobulins produced by the host can be precipitated, isolated and purified by routine methods, including affinity purification.

Preferably, the antibody according to the present invention is a monoclonal antibody. For production of monoclonal antibodies, appropriate animals will be selected and the desired immunization protocol followed. Preferred animals for production of monoclonal antibodies are for example rat and mouse. After the appropriate period of time, the spleens of these animals are excised and individual spleen cells are fused, typically, to immortalized myeloma cells under appropriate selection conditions. Thereafter, the cells are clonally separated and the supernatants of each clone are tested for the production of an appropriate antibody specific for the desired region of the antigen. Techniques for producing antibodies are well known in the art. See, e.g., Goding et al., Monoclonal Antibodies: Principles and Practice (2d ed.) Acad. Press, N.Y., and Harlow and Lane, Antibodies: A Laboratory Manual, Cold Spring Harbor Laboratory, New York (1988). Other suitable techniques involve the in vitro exposure of lymphocytes to the antigenic polypeptides or alternatively, to selection of libraries of antibodies in phage or similar vectors. Huse et al., Generation of Large Combinatorial Library of the Immunoglobulin Repertoire in Phage Lambda, Science 246:1275-1281 (1989). Monoclonal antibodies with affinities of 10⁸ liters/mole, preferably 10⁹ to 10¹⁰ or stronger, will be produced by these methods.

In general, antibodies of the invention can be prepared by techniques generally known in the field. Preferred antibodies are generated to a purified sample of peptide antigen. Suitable peptide antigens for making such antibodies are disclosed throughout the application including Examples 6-7, below. For some applications it may be as useful or more useful to employ monoclonal antibodies. See generally Harlow, E and D. Lane in Antibodies: A Laboratory Manual, Cold Spring Harbor, N.Y. (disclosing methods for making and using polyclonal and monoclonal antibodies).

For example, antibodies can be prepared by immunizing a mammal with a purified or semi-purified sample of the peptide antigen as provided herein, alone or complexed with a carrier. Suitable mammals include typical laboratory animals such as sheep, goats, rabbits, guinea pigs, rats and mice. Rats and mice, especially mice, are preferred for obtaining monoclonal antibodies. The antigen can be administered to the mammal by any of a number of suitable routes such as subcutaneous, intraperitoneal, intravenous, intramuscular or intracutaneous injection. The optimal immunizing interval, immunizing dose, etc. can vary within relatively wide ranges and can be determined empirically based on this disclosure. Typical procedures involve injection of the antigen several times over a number of months. Antibodies are collected from serum of the immunized animal by standard techniques and screened to find antibodies specific for the peptide or peptide conjugate antigen used. Monoclonal antibodies can be produced in cells which produce antibodies and those cells used to generate monoclonal antibodies by using standard fusion techniques for forming hybridoma cells. See G. Kohler, et al., Nature, 256:456 (1975). Typically this involves fusing an antibody producing cell with an immortal cell line such as a myeloma cell to produce the hybrid cell. Alternatively, monoclonal antibodies can be produced from cells by the method of Huse, et al., Science, 256:1275 (1989).

See also Harlow, E. and D. Lane, supra, for additional information relating to making and using polyclonal and monoclonal antibodies. A variety of suitable antibody purification strategies are reported which can be used in accord with this invention.

In embodiments in which monoclonal antibodies are desired, one suitable protocol provides for intraperitoneal immunization of a mouse with a composition comprising purified peptide conducted over a period of about two to seven months. Spleen cells then can be removed from the immunized mouse. Sera from the immunized mouse is assayed for titers of antibodies specific for a particular peptide prior to excision of spleen cells. The excised mouse spleen cells are then fused to an appropriate homogenic or heterogenic (preferably homogenic) lymphoid cell line having a marker such as hypoxanthine-guanine phosphoribosyltransferase deficiency (HGPRT-) or thymidine kinase deficiency (TK-). Preferably a myeloma cell is employed as the lymphoid cell line. Myeloma cells and spleen cells are mixed together, e.g. at a ratio of about 1 to 4 myeloma cells to spleen cells. The cells can be fused by the polyethylene glycol (PEG) method. See G. Kohler, et al., Nature, supra. The thus cloned hybridoma is grown in a culture medium, e.g. RPMI-1640. See G. E. More, et al., Journal of American Medical Association, 199:549 (1967). Hybridomas, grown after the fusion procedure, are screened such as by radioimmunoassay or enzyme immunoassay for secretion of antibodies that bind specifically to the antigen employed e.g. antibodies are selected that bind to the purified peptide conjugate but not to other unrelated control peptides. Preferably an ELISA or related immunological assay is employed for the screen. Hybridomas that show positive results upon such screening can be expanded and cloned by limiting dilution method. Further screens are preferably performed to select antibodies that can bind to peptide conjugate in solution. The isolated antibodies can be further purified by nearly any suitable immunological technique including affinity chromatography.

The molecular weight of the antibodies of the invention will vary depending on several factors such as the intended use and whether the antibody includes a conjugated or recombinantly fused toxin, pharmaceutical, or detectable label or the like. In general, an antibody of the invention will have a molecular weight of between approximately 20 to 150 kDa. Such molecular weights can be readily determined by molecular sizing methods such as SDS-PAGE gel electrophoresis followed by protein staining or Western blot analysis.

Preferred peptides for making polyclonal antibodies of the invention are disclosed eg., in Examples 7-8 below.

The antibodies according to the present invention generated can be used for a number of purposes, e.g., as probes in immunoassays, for inhibiting interaction between guanylin and its receptor, in diagnostic or therapeutic applications. These applications are discussed in greater detail herein below.

The antibodies of the present invention can be used with or without modification. The antibodies could comprise a labelling group which is attached to the antibody, either covalently or non-covalently. Such a labelling group is a substance which provides an either directly or indirectly detectable signal and include those that are well known in the art. For example the labelling group can be selected from the group consisting of: a fluorescent label, a radioactive label, a bioactive label and a heavy metal.

Additionally, in one embodiment the antibodies of the present invention may be chimeric, human-like or humanised, in order to reduce their potential antigenicity, without reducing their affinity for their target. Chimeric, human-like and humanized antibodies have generally been described in the art. Generally, such chimeric, human-like or humanised antibodies comprise hypervariable regions, e.g., complementarity determining regions (CDRs) from a mammalian animal, i.e., a mouse, and a human framework region. See, e.g., Queen, et al., Proc. Nat'l Acad. Sci. USA 86:10029 (1989), Verhoeyan, et al., Science 239:1534-1536 (1988). By incorporating as little foreign sequence as possible in the hybrid antibody, the antigenicity is reduced. Preparation of these hybrid antibodies may be carried out by methods well known in the art.

In one embodiment said antibody is isolated. Procedures for isolation of antibodies are well known to the person skilled in the art and they could for example be affinity-purification, Protein-A or Protein G based purification.

In one embodiment of the present invention the antibody is only a fragment of an antibody. Antibody fragments retaining useful specific binding properties can be (Fab)₂, Fab, Fv, VH or Vk fragments. These can for example be derived from an intact reshaped antibody, for example by protease digestion, or produced as such by genetic engineering.

Preferably, the antibody or fragment of an antibody according to the present invention specifically binds to a Guanylyl Cyclase C polypeptide. More preferably, the antibody or the fragment of an antibody specifically binds to a polypeptide from the extra cellular domain of Guanylyl Cyclase C. Yet more preferably, the antibody or the fragment of an antibody specifically binds to the ligand binding domain of Guanylyl Cyclase C.

In one preferred embodiment the antibody or fragment of an antibody specifically binds to a Guanylyl Cyclase C polypeptide and preferably said polypeptide comprises the amino acid sequence Xaa-Pro-Xaa-Phe-Xaa-Trp. More preferably said polypeptide comprise an amino acid sequence selected from the group consisting of: a) Ser-Pro-Thr-Phe-Ile-Trp-Lys; b) Ser-Pro-Thr-Phe-Thr-Trp-Lys; c) His-Pro-Thr-Phe-Thr-Trp-Lys; d) Ser-Pro-Asn-Phe-Ile-Trp-Lys; and e) Ser-Pro-Thr-Phe-Ile-Trp.

Preferably the antibody binds to an epitope comprising or consisting of one or more of the sequences: Ser-Pro-Thr-Phe-Ile-Trp-Lys; b) Ser-Pro-Thr-Phe-Thr-Trp-Lys; c) His-Pro-Thr-Phe-Thr-Trp-Lys; d) Ser-Pro-Asn-Phe-Ile-Trp-Lys; and e) Ser-Pro-Thr-Phe-Ile-Trp.

In one invention embodiment, the antibody has an IgG serotype. It can be polyclonal or monoclonal as desired. A particular antibody of interest is polyclonal and specifically binds one of the following peptides: H-SPNFIWKC—NH₂ (Compound 11) or H-SPNFITKC—NH₂ (Compound 11a).

In another aspect, the present invention relates to an inhibitor that is capable of associating with guanylin, such as a competitive inhibitor of the guanylin-GC-C binding. In one preferred aspect, such an inhibitor is a polypeptide or a peptide, such as an oligomer. Preferred association is by non-covalent binding eg., hydrogen bonding and/or Van der Waals interaction.

Such a polypeptide may be of any length suitable for the application. For example such a peptide comprises at least 3 amino acids, such as at least 5 amino acids, for example at least 7 amino acids, such as at least 10 amino acids, for example at least 15 amino acids, such as at least 20 amino acids, for example at least 30 amino acids. In one preferred embodiment the polypeptide comprise approximately 15 amino acids. In another preferred embodiment the polypeptide is for example heptapeptide, such as a hexapeptide or for example a pentapeptide.

In particular preferred aspects, the polypeptides of the invention will have an amino acid sequence that is related to or derived from the amino acid sequence of GC-C (Schulz et al., 1990). More preferably, the polypeptides of the invention will have an amino acid sequence that is related to or derived from the amino acid sequence of the extracellular domain of GC-C. Yet more preferably, the polypeptides of the invention will have an amino acid sequence that is related to or derived from the amino acid sequence the amino acid sequence of the ligand binding domain of GC-C.

Even more preferably, the polypeptide comprises the amino acid sequence Xaa-Pro-Xaa-Phe-Xaa-Trp, wherein Xaa may be any amino acid. Most preferably, the polypeptide comprises an amino acid sequence selected from the group consisting of: a) Ser-Pro-Thr-Phe-Ile-Trp-Lys; b) Ser-Pro-Thr-Phe-Thr-Trp-Lys; c) His-Pro-Thr-Phe-Thr-Trp-Lys; d) Ser-Pro-Asn-Phe-Ile-Trp-Lys; and e) Ser-Pro-Thr-Phe-Ile-Trp. In another embodiment the polypeptide comprise sequences that are substantially homologues an amino acid sequence selected from the group consisting of: a) Ser-Pro-Thr-Phe-Ile-Trp-Lys; b) Ser-Pro-Thr-Phe-Thr-Trp-Lys; c) His-Pro-Thr-Phe-Thr-Trp-Lys; d) Ser-Pro-Asn-Phe-Ile-Trp-Lys; and e) Ser-Pro-Thr-Phe-Ile-Trp having binding affinity for guanylin.

In one embodiment of the present invention the polypeptide is a heptapeptide comprising an amino acid sequence selected from the group consisting of: a) Ser-Pro-Thr-Phe-Ile-Trp-Lys; b) Ser-Pro-Thr-Phe-Thr-Trp-Lys; c) His-Pro-Thr-Phe-Thr-Trp-Lys; and d) Ser-Pro-Asn-Phe-Ile-Trp-Lys. In another embodiment the heptapeptide comprise sequences that are substantially homologues with an amino acid sequence selected from the group consisting of a) Ser-Pro-Thr-Phe-Ile-Trp-Lys; b) Ser-Pro-Thr-Phe-Thr-Trp-Lys; c) His-Pro-Thr-Phe-Thr-Trp-Lys; and d) Ser-Pro-Asn-Phe-Ile-Trp-Lys having binding affinity for guanylin.

In another embodiment of the present invention the polypeptide is a hexapeptide comprising the amino acid sequence Ser-Pro-Thr-Phe-Ile-Trp. In another embodiment the hexapeptide comprise sequences that are substantially homologues with the sequence Ser-Pro-Thr-Phe-Ile-Trp having binding affinity for guanylin.

The term “substantially homologous” when referring to polypeptides, refers to the homology of two amino acid sequences which, when optimally aligned, are at least about 75% homologous, preferably at least about 85% homologous more preferably at least about 90% homologous, and still more preferably at least about 95% homologous. Optimal alignment of sequences for aligning a comparison window may be conducted by the local homology algorithm of Smith and Waterman (1981) Adv. Appl. Math. 2:482, by the homology alignment algorithm of Needleman and Wunsch (1970) J. Mol. Biol. 48:443, by the search for similarity method of Pearson and Lipman (1988) Proc. Natl. Acad. Sci. (USA) 85:2444, or by computerized implementations of these algorithms (GAP, BESTFIT, FASTA, and TFASTA in the Wisconsin Genetics Software Package Release 7.0, Genetics Computer Group, 575 Science Dr., Madison, Wis.).

A further aspect of the present invention relates to an inhibitor as defined above that is capable of associating with Guanylyl Cyclase C (GC-C), wherein said association results in no stimulation of and/or cessation of catalyses of synthesis of cGMP by Guanylyl Cyclase C. Preferably such an inhibitor is an antagonist of Guanylin. It is preferred that the inhibitor upon association with Guanylyl Cyclase C sterically inhibits association of Guanylyl Cyclase C with Guanylin or with a guanylin-like comound.

Determination of whether an inhibitor is capable of associating with GC-C is preferably done by determination of the binding affinity between said inhibitor and GC-C. One preferred method for determination of binding affinity is described in example 1, however any method known to the person skilled in the art may be employed.

Topoisomers

Only one of the two naturally occuring Guanylin topoisomers activates the GC-C receptor, thus revealing the importance of a highly defined ligand structure for the biological activity of guanylin. Accordingly, in one particularly interesting embodiment of the present invention the inhibitor is an inactive Guanylin topoisomer, optionally modified to reduce or eliminate conversion between the topoisomers at physiological conditions.

The Guanylin topoisomer could be the naturally occuring inactive Guanylin topoisomer. However, it could also be a synthetic Guanylin topoisomer. In particular the inactive guanylin toposiomer may be modified, such as modified by adding at least one group and/or amino acid to the C-terminal end of the 15 residues Guanylin peptide thereby reducing interconversion between the two topoisomers. Hence, especially interesting Guanylin topoisomers comprise a C-terminally elongation of the 15 amino acid Guanylin peptide. Such C-terminal elongation could be one amino acid long, such as at least 2 amino acids long, for example at least 5 amino acids long, such as at least 10 amino acids long, for example at least 15 amino acids long, such as at least 20 amino acids long, for example at least 30 amino acids long. In one embodiment said C-terminal elongation comprise one amino acid, said one amino acid for example being Leu.

Examples of such C-terminal elongation with Leu are:

Pro-Asn-Thr-Cys-Glu-Ile-Cys-Ala-Tyr-Ala-Ala-Cys-Thr-Gly-Cys-Leu

Guanylin derived polypeptides

In another embodiment of the present invention, the inhibitor is a modified polypeptide (analogue) derived from a naturally-occuring guanylin sequence:

| | |
|---|---|
| Pro-Asn-Thr-Cys-Glu-Ile-Cys-Ala-Tyr-Ala-Ala-Cys-Thr-Gly-Cys | (Rat) |
| Pro-Asn-Thr-Cys-Glu-Ile-Cys-Ala-Tyr-Ala-Ala-Cys-Thr-Gly-Cys | (Mouse) |
| Pro-Gly-Thr-Cys-Glu-Ile-Cys-Ala-Tyr-Ala-Ala-Cys-Thr-Gly-Cys | (Human) |
| Pro-Ser-Thr-Cys-Glu-Ile-Cys-Ala-Tyr-Ala-Ala-Cys-Ala-Gly-Cys | (Pig) |
| Ser-His-Thr-Cys-Glu-Ile-Cys-Ala-Phe-Ala-Ala-Cys-Ala-Gly-Cys | (Opossum) |
| Pro-Ser-Thr-Cys-Glu-Ile-Cys-Ala-Tyr-Ala-Ala-Cys-Ala-Gly-Cys | (Guinea pig) |

Preferred guanylin analogues of the invention can be derived by substituting, deleting and/or adding at least one amino acid to a naturally-occuring guanylin in which the modified polypeptide has at least one disulfide bridge, such as preferably two disulfide bridges. Preferably said disulfide bridges are arranged in a way, such as they are between Cys 4 and Cys 12 and between Cys 7 and Cys 15, the numbering according to the position in the guanylin wild type sequence. Such a polypeptide should be able to inhibit association between active guanylin and GC-C, and thereby indirectly inhibiting synthesis of cGMP. The modification may be constituted of a combination of substitution and deletion and/or addition, as well as a combination of deletion of amino acids in one part of the sequence combined with addition and/or substitution of amino acids in another part of the sequence.

When the amino acid sequence comprises a substitution of one amino acid for another, such a substitution may be a conservative amino acid substitution or a non-conservative substitution. Guanylin according to the present invention may comprise more than one such substitution, such as e.g. two amino acid substitutions, for example three or four amino acid substitutions, such as five or six amino acid substitutions, for example seven or eight amino acid substitutions, such as from 10 to 14 amino acid substitutions. Accordingly, inactive Guanylin derived poly-peptides according to the invention may comprise a plurality of substitutions introduced independently of one another.

Preferably, the inactive polypeptide derived from guanylin comprise the guanylin amino acid sequence as outlined above, wherein any one or more of the amino acids has been substituted by Ala, such as one of the following sequences:

Ala-Asn-Thr-Cys-Glu-Ile-Cys-Ala-Tyr-Ala-Ala-Cys-Thr-Gly-Cys
Pro-Ala-Thr-Cys-Glu-Ile-Cys-Ala-Tyr-Ala-Ala-Cys-Thr-Gly-Cys
Pro-Asn-Ala-Cys-Glu-Ile-Cys-Ala-Tyr-Ala-Ala-Cys-Thr-Gly-Cys
Pro-Asn-Thr-Ala-Glu-Ile-Cys-Ala-Tyr-Ala-Ala-Cys-Thr-Gly-Cys
Pro-Asn-Thr-Cys-Ala-Ile-Cys-Ala-Tyr-Ala-Ala-Cys-Thr-Gly-Cys
Pro-Asn-Thr-Cys-Glu-Ala-Cys-Ala-Tyr-Ala-Ala-Cys-Thr-Gly-Cys
Pro-Asn-Thr-Cys-Glu-Ile-Ala-Ala-Tyr-Ala-Ala-Cys-Thr-Gly-Cys
Pro-Asn-Thr-Cys-Glu-Ile-Cys-Ala-Ala-Ala-Ala-Cys-Thr-Gly-Cys
Pro-Asn-Thr-Cys-Glu-Ile-Cys-Ala-Tyr-Ala-Ala-Ala-Thr-Gly-Cys
Pro-Asn-Thr-Cys-Glu-Ile-Cys-Ala-Tyr-Ala-Ala-Cys-Ala-Gly-Cys
Pro-Asn-Thr-Cys-Glu-Ile-Cys-Ala-Tyr-Ala-Ala-Cys-Thr-Ala-Cys
Pro-Asn-Thr-Cys-Glu-Ile-Cys-Ala-Tyr-Ala-Ala-Cys-Thr-Gly-Ala

The polypeptide derived from guanylin may comprise addition and/or deletion of an amino acid, such as addition and/or deletion of from 2 to preferably 10 amino acids, such as from 2 to 8 amino acids, for example from 2 to 6 amino acids, such as from 2 to 4 amino acids. However, additions of more than 10 amino acids, such as additions from 10 to 200 amino acids, are also comprised within the present invention. Amino acid deletions, additions and substitutions in accord with the invention can be contiguous or non-contiguous as needed.

In one embodiment the inactive polypeptides derived from guanylin comprise the guanylin amino acid sequence as outlined above, wherein one or more of the N terminal or C-terminal amino acids have been deleted. The following sequences are examples of such polypeptides:

Asn-Thr-Cys-Glu-Ile-Cys-Ala-Tyr-Ala-Ala-Cys-Thr-Gly-Cys
Thr-Cys-Glu-Ile-Cys-Ala-Tyr-Ala-Ala-Cys-Thr-Gly-Cys
Cys-Glu-Ile-Cys-Ala-Tyr-Ala-Ala-Cys-Thr-Gly-Cys

In one preferred embodiment the polypeptides derived from Guanylin comprise an addition of at least one amino acid. Preferably, said at least one amino acid is Leu, more preferably said amino acid is one Leu residue. Such an addition may be N-terminal or C-terminal, preferably the addition is C-terminal.

Inactive polypeptides sharing at least some homology with guanylin are to be considered as falling within the scope of the present invention when they are at least about 40 percent homologous with guanylin, such as at least about 50 percent homologous, for example at least about 60 percent homologous, such as at least about 70 percent homologous, for example at least about 75 percent homologous, such as at least about 80 percent homologous, for example at least about 85 percent homologous, such as at least about 90 percent homologous, for about 90 percent homologous with guanylin.

In addition to the polypeptides described herein, sterically similar compounds may be formulated to mimic the key portions of the polypeptide structure and that such compounds may also be used in the same manner as the polypeptides of the invention. This may be achieved by techniques of modelling and chemical designing known to those of skill in the art. For example, esterification and other alkylations may be employed to modify the amino terminus of, e.g., a di-arginine peptide backbone, to mimic a tetrapeptide structure. It will be understood that all such sterically similar constructs fall within the scope of the present invention.

Peptides with N-terminal alkylations and C-terminal esterifications are also encompassed within the present invention. Inactive Guanylin equivalents also comprise glycosylated and/or phosphorylated and/or sulphated and covalent or aggregative conjugates froms of Guanylin, including dimers or unrelated chemical moieties. Such inactive equivalents are prepared by linkage of functionalities to groups which are found in fragment including at any one or both of the N- and C-termini, by means known in the art.

In yet another aspect of the present invention the amino acid sequence of the inhibitor comprises a modification of at least one amino acid, such as a halogenated derivative of Guanylin exhibiting unchanged specific binding to the GC-C receptor while the biological activity is significantly reduced. Halogen may be selected from F. Cl, Br, and I, preferably Br and I, such as iodinated[Tyr(3,5-diiodo)⁹]guanylin as well as iodonated[Tyr(3-iodo)⁹]guanylin.

In particular such inhibitors are polypeptides of the general sequence:

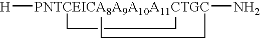

wherein A₈, A₉, A₁₀ and A₁₁ could be any amino acid.

Preferably, A₈ is an aliphatic amino acid. More preferably A₈ is selected from the group consisting of Gly, Ala, Leu, Ile and Val. Preferably A₉ is selected from the group consisting of Asn and Gln or A₉ is an aromatic amino acid. A₉ is optionally substituted, such as substituted with one or more groups selected from C(1-4)-alkyl, C(1-4)-alkenyl, halogen, cyano, isocyano, isothiocyano, thiocyano, sulfamyl, C(1-4) alkylthio, mono- or di-C-(1-4)-alkyl-amino, hydroxy, C(1-4alkoxy, aryl, heteroaryl, aryloxy, carboxy, C(1-4)-alkoxycarbonyl, C(1-4)-alkylcarbonyloxy, aminocarbonyl, mon- or di-C(1-4)-alkyl-aminocarbonyl, mono- or di-C(1-4)-alkyl-amino, mono- or di-C(1-4alkyl-amino-C(1-4)-alkyl, C(1-4)-alkylcarbonylamino, sulfono, and sulfino; and where an aryl or a heteroaryl group may be further substituted with one or more groups selected from C(1-4)-alkyl, C(1-4)-alkenyl, nitro, amino halogen, cyano, isocyano, isothiocyano, thiocyano, sulfamyl, C(1-4) alkylthio, mono- or di-C-(1-4)-alkyl-amino, hydroxy, C(1-4)-alkoxy, aryloxy, carboxy, C(1-4)-alkoxycarbonyl, C(1-4)-alkylcarbonyloxy, aminocarbonyl, mono- or di-C(1-4)-alkyl-aminocarbonyl, mono- or di-C(1-4)-alkyl-amino, mono- or di-C(1-4)alkyl-amino-C(1-4alkyl, C(1-4)-alkylcarbonylamino, sulfono, and sulfino. The halogen can be selected from the group consisting of F, Cl, Br, and I. Preferably the halogen is selected from the group consisting of F and Cl. Preferably, A₁₀ an aliphatic amino acid. More preferably, A₁₀ is selected from the group consisting of Gly, Ala, Leu, Ile and Val. Preferably, A₁₁ is an aliphatic aminoacid. More preferably, A₁₁ is selected from the group consisting of Gly and Ala. Disulfide bridges are shown with lines.

In another embodiment of the present invention the inhibitor is a polypeptide of the general sequence

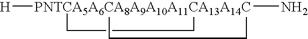

wherein A₅, A₆, A₈, A₉, A₁₀, A₁₁, A₁₃ and A₁₄ could be any amino acid.

When, A₅ is Glu preferably A₆ is not Ile, and/or A₈ is not Ala and/or A₉ is not Tyr and/or A₁₀ is not Ala and/or A₁₁ is not Ala and/or A₁₃ is not Thr and/or A₁₄ is not Gly, or

when A₆ is Ile, preferably A₅ is not Glu and/or A₈ is not Ala and/or A₉ is not Tyr and/or A₁₀ is not Ala and/or A₁₁ is not Ala and/or A₁₃ is not Thr and/or A₁₄ is not Gly, or

When A₈ is Ala preferably A₅ is not Glu and/or A₉ is not Ile, and/or A₉ is not Tyr and/or A₁₀ is not Ala and/or A₁₁ is not Ala and/or A₁₃ is not Thr and/or A₁₄ is not Gly, or

When A₉ is Tyr, preferably A₅ is not Glu and/or A₆ is not Ile, and/or A₈ is not Ala and/or A₁₀ is not Ala and/or A₁₁ is not Ala and/or A₁₃ is not Thr and/or A₁₄ is not Gly

When or A₁₀ is Ala, preferably A₅ is not Glu and/or A₆ is not Ile, and/or A₈ is not Ala and/or A₉ is not Tyr and/or A₁₁ is not Ala and/or A₁₃ is not Thr and/or A₁₄ is not Gly

When A₁₁ is Ala, preferably A₅ is not Glu and/or A₆ is not Ile, and/or A₈ is not Ala and/or A₉ is not Tyr and/or A₁₀ is not Ala and/or A₁₃ is not Thr and/or A₁₄ is not Gly

When A₁₃ is Thr, preferably A₅ is not Glu and/or A₆ is not Ile and/or A₈ is not Ala and/or A₉ is not Tyr and/or A₁₀ is not Ala and/or A₁₁ is not Ala and/or A₁₄ is not Gly,

When A₁₄ is Gly preferably A₅ is not Glu and/or A₆ is not Ile and/or A₈ is not Ala and/or A₉ is not Tyr and/or A₁₀ is not Ala and/or A₁₁ is not Ala and/or A₁₃ is not Thr,

In one embodiment A₅, A₆, A₈, A₉, A₁₀, A₁₃ and A₁₄ are all Ala. Disulfide bridges are shown with lines.

Additionally preferred guanylins and guanylin analogues are shown below in Table 1:

TABLE 1¹

| Com- | |
|---|---|
| pound | |
| # | Common Name/Sequence |
| 1 | Mouse-Guanylin-NH2, |
| | H-PNTC*EICAYAAC*TGC-NH2 (double oxidised C to C and |
| | C* to C*) |
| 2 | Guanylin-K₆-NH₂(rat/mouse): H- |
| | PNTC*EICAYAAC*TGCKKKKKK-NH₂ |
| | (double oxidised C to C and C* to C*) |
| 3 | H-SPNFIWK-NH₂ |
| 3A | H-SPNFITK-NH₂ |
| 4 | cyclo(GEIGAYAAGTGQ) |
| 5 | des-Pro¹, des-Asn²[Phe⁹]Guanylin-L-NH₂ (rat/mouse) |
| 6 | des-Pro¹, des-Asn²[2-Nal⁹]Guanylin-L-NH₂ (rat/mouse) |
| 7 | des-Pro¹, des-Asn²Guanylin-LKK-NH₂ mono oxidised(1-3) |
| | (rat/mouse) |
| 8 | des-Pro¹, des-Asn²Guanylin-LKK-NH₂ fully oxidised(1-3, 2-4) |
| | (rat/mouse): H-TCEICAYAACTGCLKK-NH₂ oxidised(1-3, |
| | 2-4) |
| 9 | Des-Pro¹, des-Asn²Guanylin-LKK-NH₂ fully oxidised(1-3, 2-4) |
| | (rat/mouse): H-TCEICAYAACTGCLKK-NH₂ oxidised(1-3, |
| | 2-4) |
| 10 | des-Pro¹, des-Asn²Guanylin-LKK-NH₂ fully oxidised(1-3, 2-4): |
| 11 | H-SPNFITKC-NH₂ |
| 11A | H-SPNFIWKC-NH₂ |
| 12 | Guanylin-Asp-Lys₆-NH₂ (rat/mouse) |
| 13 | Mouse-Guanylin-Asp-OH |

¹The phrase “rat/mouse” means that the compound has essentially the same structure in mice and rats.

Prodrugs

This invention also encompasses prodrug derivatives of the compounds for example the polypeptides contained herein. The term “prodrug” refers to a derivative of a parent drug molecule that requires biotransformation, either spontaneous or enzymatic, within the organism to release the parent drug. Prodrugs are variations or derivatives of the compounds for example the polypeptides of this invention which have groups cleavable under metabolic conditions. Prodrugs become the compounds for example the polypeptides of the invention which are pharmaceutically active in vivo, when they undergo solvolysis under physiological conditions or undergo enzymatic degradation. Prodrug compounds of this invention may be called single, double, triple etc., depending on the number of biotransformation steps required to release the active drug within the organism, and indicating the number of functionalities present in a precursor-type form. Prodrug forms often offer advantages of solubility, tissue compatibility, or delayed release in the mammalian organism. Furthermore, prodrugs can be advantageous due to facilitated synthesis and/or purification of the prodrug compared to the polypeptide according to the invention.

One preferred example of a prodrug is the addition of at least 4 Lys residues to a polypeptide according to the present invention, such as 4 Lys residues, for example 5 Lys residues, such as at least 6 residues. Preferably, the addition is 6 Lys residues. Such an addition of Lys residues greatly facilitates the synthesis and subsequent purification of the polypeptide. The addition of Lys residues may be either N-terminal or C-terminal. Preferably, such addition is C-terminal.

Cyclic polypeptides

In one particularly interesting embodiment the present invention relates to cyclic polypeptides. One example of a cyclic polypeptide within the scope of the present invention is a cyclic polypeptide with the general sequence:

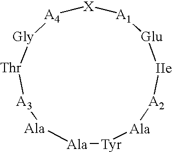

wherein A₁, A₂, A₃, and A₄ could be any amino acid.

Preferably, A₁ is selected from the group consisting of Gly, Cys, Ser, Lys, Lysine analogues, Thr, Glu and Ala. Preferably, A₂ is selected from the group consisting of Gly and Ala. Preferably, A₃ is selected from the group consisting of Gly and Ala. Preferably, A₄ is selected from the group consisting of Gly, Cys, Ser, Lys, Lysine analogues, Thr, Glu and Ala. X is a bond, such as a bond selected from thioamide bond, thioester bond, hydrazide bond, amide bond, a reduced amide bond, an ester bond, or a disulphide bond, or a spacer, such as beta-Ala, 4-aminobutane acid, 5-aminopentane acid, and hydroxy acids.

Another example of a cyclic polypeptide is the cyclic polypeptide with the sequence:

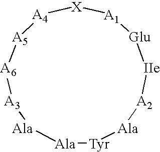

wherein A₁, A₂, A₃, A₄, A₅, and A₆ could be any amino acid. Preferably, A₁ is selected from the group consisting of Gly, Cys, Ser, Lys, Lysine analogues, Thr, Glu and Ala. Preferably, A₂ is selected from the group consisting of Gly and Ala. Preferably, A₃ is selected from the group consisting of Gly and Ala. Preferably, A₄ is selected from the group consisting of Gly, Cys, Ser, Lys, Lysine analogues, Thr, Glu and Ala. Preferably, A₅ is selected from the group consisting of Ser and Ala. Preferably, A₆ is selected from the group consisting of Ser and Ala. X is a bond, such as a bond selected from thioamide bond, thioester bond, hydrazide bond, amide bond, a reduced amide bond, an ester bond, or a disulphide bond, or a spacer, such as beta-Ala, 4-aminobutane acid, 5-aminopentane acid, and hydroxy acids.

Yet another example of a cyclic polypeptide is the cyclic polypeptide with the sequence:

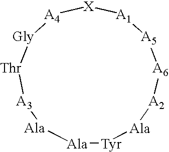

wherein A₁, A₂, A₃, A₄, A₅, and A₆ could be any amino acid. Preferably, A₁ is selected from the group consisting of Gly, Cys, Ser, Lys, Lysine analogues, Thr, Glu and Ala. Preferably, A₂ is selected from the group consisting of Gly and Ala. Preferably, A₃ is selected from the group consisting of Gly and Ala. Preferably, A₄ is selected from the group consisting of Gly, Cys, Ser, Lys, Lysine analogues, Thr, Glu and Ala. Preferably, A₅ is selected from the group consisting of Asp, Asn, Gln, Leu, Val, Ala and Gly. Preferably, A₆ is selected from the group consisting of Asp, Asn, Gln, Leu, Val, Ala and Gly. X is a bond, such as a bond selected from thioamide bond, thioester bond, hydrazide bond, amide bond, a reduced amide bond, an ester bond, or a disulphide bond, or a spacer, such as beta-Ala, 4-aminobutane acid, 5-aminopentane acid, and hydroxy acids.

An additional example of a cyclic polypeptide, is the cyclic polypeptide with the sequence:

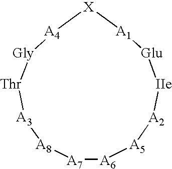

wherein A₁, A₂, A₃, A₄, A₅, A₆, A₇, and A₈ independently may be any amino acid. Preferably, A₁ is selected from the group consisting of Gly, Cys, Ser, Lys, Lysine analogues, Thr, Glu and Ala. Preferably, A₂ is selected from the group consisting of Gly and Ala. Preferably, A₃ is selected from the group consisting of Gly and Ala. Preferably, A₄ is selected from the group consisting of Gly, Cys, Ser, Lys, Lysine analogues, Thr, Glu and Ala. Preferably, A₅ is selected from the group consisting of Ala, Gly, Val, Leu, Ile, Asn, Gln, Tyr, His and Trp. Preferably, A₆ is selected from the group consisting of Ala, Gly, Val, Leu, Ile, Asn, Gln, Tyr, His and Trp. Preferably, A₇ is selected from the group consisting of Ala, Gly, Val, Leu, Ile, Asn, Gln, Tyr, His and Trp. Preferably, A₈ is selected from the group consisting of Ala, Gly, Val, Leu, Ile, Asn, Gln, Tyr, His and Trp. X is a bond, such as a bond selected from thioamide bond, thioester bond, hydrazide bond, amide bond, a reduced amide bond, an ester bond, or a disulphide bond, or a spacer, such as beta-Ala, 4-aminobutane acid, 5-aminopentane acid, and hydroxy acids.

In one invention embodiment, the cyclic polypeptide has the following structure:

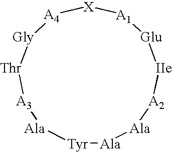

in which A1, A2, A3 and A4 are each as defined above.

Pharmaceutical composition

Symptoms of Ménière's disease can be selected from the group consisting of: fluctuating hearing levels, sensation of fullness in the ear, roaring tinnitus and episodic vertigo.

The treatment according to the present invention may be ameliorating, curative and/or prophylactic, with respect to one or more of the symptoms of Ménière's disease as well as the symptoms individually whether they are connected to Ménière's disease. In one embodiment the treatment is effective, either ameliorating, curative and/or prophylactic against all the symptoms of Ménière's disease.

The individual to be treated could be any animal including a human being, in need of such treatment. Preferably, such an individual is a human being.

The preferred mode of administration depends upon the compound to be administered and the individual to be treated. For example the administration could be oral, subcutaneous, parenteral (Intravenous, Intraperitoneal), intramuscular, intralipomateous, rectal, epidural, intratracheal, dermal, vaginal, buccal, occurlarly, directly into the brain, pulmonary administration or topical administration in the ear canal. For most purposes according to the present invention, either oral or parenteral administration are usefull.

Pharmaceutical compositions according to the present invention comprise an inhibitor, in particular said inhibitor is capable of inhibiting the interaction between active Guanylin and GC-C. Furthermore such pharmaceutical compositions may comprise, a pharmcaeutical acceptable carrier and/or diluent. Such compositions may be in a form adapted to oral, subcutaneous, parenteral (Intravenous, Intraperitoneal), intramuscular, rectal, epidural, intratracheal, dermal, vaginal, buccal, occurlarly, direct brain or pulmonary administration, or injection of composition through the eardrum into the middle ear. The active compound (peptide) is then taken up by the inner ear. The composition is preferably in a form adapted to subcutaneous, intravenous or oral administration, and such compositions may be prepared in a manner well-known to the person skilled in the art, e.g. as generally described in “Remington's Phamaceutical Sciences”, 17. Ed. Alfonso R. Gennaro (Ed.) mark Publishing Company, Eason Pa., USA, 1985 and more recent editions and in the monographs in the Drugs and the Pharmaceutical sciences” series, Marcel Dekker. The compositions may appear in conventional forms, for example solutions and suspensions for injections, capsules and tablets, preferably in the form of enteric formulations.

The pharmaceutical carrier and/or diluent employed may be a conventional solid or liquid carrier. Examples of solid carriers are lactose, terra alba, sucrose, cyclodextrin, talc, gelatin, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene and water.

Similarly, the carrier and/or dileunt my include sustained release material known to the person skilled in the art, such as glyceryl monostereate or glyceryl distereate, alone or mixed with wax.

If a solid carrier is used for oral administration, the preparation may be tabletted, placed in a hard gelatin capsule in powder or pellet form or it can be in the form of a troche or lozenge. The amount of solid carrier will vary widely but will usually be from about 25 mg to about 1 g.

If a liquid carrier is used, the preparation may be in the form of a syrup, emulsion, soft gelatin capsule or sterile injectable liquid such as an aqueous or non-aqueuos liquid suspension or solution.

The composition may also be in the form suited for local or systemic injection or infusion and may, as such be formulated with sterile water or an isotonic saline or glucose solution. The compositions may be sterilised by conventional sterilisation techniques which are well known to the person skilled in the art. The resulting aqueous solutions may be packaged for use or filtered under aseptic conditions and lyophilised, the lyophilised preparation being combined with the sterile aqueous solution prior to administration. The composition may contain pharmaceutically acceptable auxiliary substances such as required to approximate physiological conditions, such as buffering agents, tonicity adjusting agents and the like for instance sodium acetate, sodium lactate, sodium chloride, potassium chloride, calcium chloride etc.

For intravenous injection multi-dose formulation may be prepared, for example as a solution of a compound of the invention in sterile isotonic saline, stored in capped vials, and if necessary a preservative is added (e.g. benzoates). Fixed dose formulation may be prepared as a solution of the compound in sterile, isotonic saline, stored in glass ampoules and if necessary filled with an inert gas. Each dose of the compound is stored dry in ampoules or capped vials, if necessary filled with inert gas. The multi-dose formulation demands the highest degree of stability of the compound. When the-stability of the compound is low fixed dose formulations can be used.

For nasal administration the preparation may contain a compound of the present invention dissolved or suspended in a liquid carrier, in particular, an aqueous carrier, for aerosol application. The carrier may contain additives such as solubilising agents, e.g., propylene glycol, surfactants such as bile acid salts or polyoxyethylene higher alcohol ethers, absorption enhancers such as lecithin (phosphatidylcholine) or cyclodextrin or preservatives such as parabines.

It is preferred that the inhibitors of the present invention are stable towards enzymatic degradation and/or stable in plasma. It may be desirable to employ various derivatives and/or chemical modifications of the polypeptides described in hereon above. Especially, such derivatives and/or chemical modification could enhance stability while retaining the essential properties of the native polypeptides as presented by the invention. Such modification could for example be C-terminal amidation or esterification, the use of d-amino acids and derivatives of natural amino acids, N-terminal structural amino acid analogues, such as phenyl acetic acid and phenyl glycine that lack a carboxylic function, and cyclic analogues.

Moreover, the small size of the peptide compounds of the invention may be an advantage for oral and nasal administration, since the relatively fast absorption via mucosal membranes compared to larger peptides minimises enzymatic degradation, specially in the duodenum and the ileum.

Furthermore, the invention relates to the use of a compound, including polypeptides, peptides, oligomers, antibodies, small molecules, small organic molecules as well as combinations thereof for the production of a pharmaceutical composition.

Also, the invention relates to the use of a compound as defined above for preparation of a pharmaceutical composition. Furthermore, the invention relates to the use of a compound as defined above for preparation of a pharmaceutical composition for the treatment of one or more of the symptoms vertigo, hearing loss and tinnitus, as well as one or more of the symptoms of Ménière's disease.

Yet another embodiment is the treatment of one or more of the symptoms vertigo, hearing loss and tinnitus, as well as one or more of the symptoms of Ménière's disease by administering to an individual in need thereof a sufficient amount of a compound as defined herein or a pharmaceutical composition as defined herein.

Definitions

Binding affinity: Determination of binding affinity of compounds according to the present invention to GC-C can be done as described in example 1.

By the phrase “specific binding” or related phrase as it relates to association between an antibody and peptide or peptide conjugate antigen is meant that the antibody forms an immune complex with the particular antigen and not with other antigens (such as related or unrelated peptide conjugates). Methods for detecting and optionally quantifying such specific binding include standard immmunoassays eg., ELISA, antibody capture and antigen capture assays. Preferred antibodies of the invention specifically bind a subject peptide or peptide conjugate antigen. See Example 7 and 8 below, for instance.

Compound which comprises Guanylin like activity: Any compound that is capable of association with GC-C in a manner, such as said association results in the stimulation of the intracellular domain of GC-C, such as the intracellular domain catalyses the synthesis of cGMP, and example of said compound being a heat-stable enterotoxin produced by E. coli.

GC-C: Guanylyl Cyclase C or guanylate cyclase C refers to the membrane associated or membrane bound receptor, also known as the particulate GC-C receptor or heat-stable enterotoxin receptor precursor (GC-C).

Inactive: A compound that do not comprise a biological activity. For example inactive Guanylin, is Guanylin that could be capable of associating with GC-C, however said association does not lead to catalysis of cGMP synthesis by the intracellular domain of GC-C.

Amino acids

Throughout the description and claims either the three letter code or the one letter code for natural amino acids are used, such as Sarcosin (Sar), alpha-Amino-iso-butanoic acid (Aib), Naphthylalanine (Nal) including 1-naphthylalanine (1Nal) and 2-naphthylalanine (2Nal), Phenylglycine Phg, 2,4-Diaminobutanoic acid (Dab), 2,3-Diaminopropanoic acid (Dapa), and Hydroxyproline (Hyp). Where the L or D form has not been specified it is to be understood that the amino acid in question has the natural L form, cf. Pure & Appl. Chem. Vol. (56(5) pp 595-624 (1984) or the D form, so that the peptides formed may be constituted of amino acids of L form, D form, or a sequence of mixed L forms and D forms

Where nothing is specified it is to be understood that the C-terminal amino acid of a polypeptide of the invention exists as the free carboxylic acid, this may also be specified as “—OH”. However, the C-terminal amino acid of a compound of the invention may be the amidated derivative, which is indicated as “—NH₂”. Where nothing else is stated the N-terminal amino acid of a polypeptide comprise a free amino-group, this may also be specified as “H—”.

Where nothing else is specified amino acid can be selected from any amino acid, whether naturally occurring or not, such as alpha amino acids, beta amino acids, and/or gamma amino acids. Accordingly, the group comprises but are not limited to: Ala, Val, Leu, Ile, Pro, Phe, Trp, Met, Gly, Ser, Thr, Cys, Tyr, Asn, Gln, Asp, Glu, Lys, Arg, His Aib, Nal, Sar, Orn, Lysine analogues DAP and DAPA, 4Hyp.

As discussed, the invention encompasses a method for making a stabilised guanylin analogue that includes a mammalian guanylin. This aspect of the invention provides many uses and advantages including assisting solid phase synthesis and purification of mammalian guanylin and analogues thereof. Typically, such a method includes reversibly adding at least one charged amino acid to the C-terminus of the guanylin, thereby making a stabilised guanylin analogue. Usually, a standard solid phase peptide synthesis method will be used to make the peptide although other methods (eg., biological synthesis such as by recombinant means) may be preferred in some instances.

More particularly, this embodiment of the method involves the addition of at least one charged amino acid which amino acid can be the same or different. Preferably, about 1 to about 25 amino acids are added to the peptide or peptide analogue, more preferably about 3 to about 10 of such amino acids with about 4 to about 8 charged amino acids being preferred for many applications. Without wishing to be bound to theory, it is believed that by adding at least one charged amino acid to the C-terminus of guanylin or analogues of guanylin, it is possible to improve the solubility of the peptide intermediates in the synthesis and to facilitate the assembling of the guanylin peptides and analogues thereof on the solid support. This feature of the invention provides advantages including making synthesis of the peptides easier and more cost effective.

In a particular invention embodiment, between from about 1 to about 25 charged amino acids are added to the guanylin. Preferred charged amino acids are selected from the group of amino acids having propensity factor of about Pα>0.57 and a propensity factor of about Pβ equal to or less than about 1.10. More specific amino acids for use in accord with the invention include lysine, arginine, aspartic acid, or glutamic acid. Such amino acids may be the same (ie. a homopolymer) or different (ie. a heteropolymer) as needed.

By way of illustration, the following amino acid sequences can be added to the guanylin C-terminus to stabilise the peptide: Asp, Asp-Asp, Asp-Asp-Asp, Lys, Asp-Lys, Asp-Lys-Lys, Asp-Lys-Lys-Lys, Asp-Lys-Lys-Lys-Lys, Asp-Lys-Lys-Lys-Lys-Lys, Asp-Lys-Lys-Lys-Lys-Lys-Lys, Lys-Lys, Lys-Lys-Lys, Lys-Lys-Lys-Lys-, Lys-Lys-Lys-Lys-Lys- or Lys-Lys-Lys-Lys-Lys-Lys. Preferred guanylin analogues are selected from the group consisting of mouse guanylin—K₆—OH/NH₂, mouse guanylin-D—K₆—OH/NH₂, mouse guanylin-D—K—OH/NH₂, mouse guanylin-D—OH/NH₂, mouse desP¹,N²-guanylin-L—K₂—OH/NH₂, mouse desP¹,N²-guanylin-L—OH/NH₂, mouse desP¹,N²-guanylin-D—K₆—OH/NH₂, mouse desP¹,N²-guanylin-D—OH/NH₂, and mouse desP¹,N²-guanylin—OH/NH₂. Additionally preferred guanylin analogues are made by one or a combination of methods as described herein.

As discussed, it is an object of the invention to provide for reversible addition of the charged amino acid(s) to the guanylin although removal of the tail is not needed to practice the invention. In cases in which such removal is desired, one or more chemical and/or biological cleaving reagents can be used to sever the charged amino acid(s) from the guanylin. For example, one or more enzymes, (eg., peptidases or proteases) can be used to cleave the tail from the guanylin peptide. Choice of a particular enzyme or group of enzymes will be guided by recognized parameters including the charged amino acid(s) used and the site(s) of cleavage desired. Separation of the charged amino acid(s) from the guanylin or analogue can be detected by one or a combination of conventional techniques including HPLC and protein gel electrophoresis. Examples of preferred enzymatic cleaving agents are provided in the examples and include proteases that cleave charged amino acid tails consisting of Asp and Lys residues.

The invention provides other uses and advantages. For example, there is recognition that guanylin and its analogues stimulate the GC-C formation of cGMP. Accordingly it is believed that it will be useful in the treatment of erectile dysfunction. It is known that enhancement of penile erection in patients with erectile dysfunction involves potentiation of the NO-stimulated cGMP signal mediating relaxation of cavernosal smooth muscle during sexual stimulation. The relaxing effect of nitric oxide on the trabecular smooth muscle is mediated through its stimulation of guanylate cyclase and the production of cyclic guanosine monophosphate (cGMP), which then function as a second messenger in this system.

The disclosures of documents mentioned herein are incorporated herein by reference.

The following non-limiting examples are illustrative of the present invention.

### EXAMPLE 1

### Screening assay

Human T84 cell line

T84 cells are grown to near confluence in DMEM medium and Ham's F-12 medium (1:1) supplemented with 5% Foetal Calf Serum (FCS), 60 mg penicillin/ml, and 100 mg streptomycin/ml in 175 cm² culture flasks (Nunc) at 37° C., 5% CO₂ and 100% humidity. Cells are harvested in ice-cold PBS and centrifuged at 100×G for 10 min at 4° C. The sedimented cells are lysed in 2.5 ml dist. H₂O/culture flask at 0° C. for 30 min and centrifuged at 50,000×G 4° C. for 45 min. The membrane pellet is taken up in binding buffer (50 mM HEPES, 1 mM EDTA, 10 mM MgCl₂, pH 7.4) supplemented with 10% sucrose and stored at −80° C. until use.

For stimulation of cGMP formation, cells are seeded in 96-well microtiter plates (Nunc) at a density of 2500 cells/well (˜7576 cells/cm²) and grown for 3 days before use under the same culture conditions mentioned above.

Binding to membranes expressing the human guanylyl cyclase (GC-C) receptor

GC-C membrane receptors (10 μgₚᵣₒₜₑᵢₙ/assay) are incubated for 60 min at 25° C. in a total volume of 100 μl binding buffer (50 mM HEPES+1 mM EDTA+10 mM MgCl₂, pH 7.4) supplemented with 1% BSA (to avoid ligand depletion) together with 1.2 nM [³H]guanylin either alone (Total binding), in the presence 10 μM guanylin (Non-specific binding) or increasing concentrations of test compounds. The binding reaction is stopped by vacuum filtration on a Packard Cell Harvester onto 96-well UniFilter^{R} GF/C™ pre-soaked at least 30 min before use in 0.5% polyethyleneimine (PEI) followed by 3 washes with ice-cold binding buffer. Filters are dried for 90 min at 60° C. before addition of 50 μl scintillation fluid (Ultima Gold cat. no. 6013329, Packard). The filter bound radioactivity (˜membrane bound) is measured in a TopCount™ (Packard Canberra) scintillation counter.

cGMP-efficacy on T84 cells

On the day of analysis growth medium is removed and the T84 cells are washed twice in Dulbeccos Phosphate Buffered Saline™ (D-PBS) containing 6 mM glucose at 37° C. Cells are then incubated at 37° C. for 40 min in the same medium supplemented with 2 mM IBMX (a phosphodiesterase inhibitor) and increasing concentrations of test compounds. The reaction is stopped by addition of ice-cold 20 μl 0.50 M HCl and incubation on ice for further 20 min. 20 μl of the acid extract is used for determination of cGMP by the FlashPlate™ technique and another 20 μl is used for determination of protein content.

Calculations

Data from the displacement experiments are fitted to the equation:

f=(Total−ns)/(1+s/IC₅₀)+ns

where Total is the total bound radioactivity at concentration s of labelled ligand, ns is non-specific binding and IC₅₀ is the concentration of test compound reducing specific binding (Total−ns) to 50% of maximum specific binding.

Data from the cGMP experiments are treated in an analogueous way and fitted to:

f=(cGMPₘₐₓ−cGMP_{basic})*s/(s+EC₅₀)+cGMP_{basic}

where cGMPₘₐₓ is maximal obtainable cGMP and cGMP_{basic} is the basic level of cellular cGMP. EC₅₀ is the concentration giving half maximum effect and s the concentration of test compound.

### EXAMPLE 2

### Stabilisation and Use of Guanylin and Guanylin Analogues

Guanylin stabilisation and structural analysis

Unlike other multiple disulfide-bridged peptides spectroscopical investigations have demonstrated that guanylin contains two structurally distinct species that differ in the backbone conformation while containing the same disulfide connectivity. The two forms are called topological stereoisomers. The two isomers exist in a dynamic equilibrium and interconvert without disulfide opening and rearrangement. Lowering the temperature to +10° C. makes it possible to separate the two isomers by HPLC. However, the rapid interconversion between the two isomers has made it impossible to isolate them in pure forms. C-terminally elongation of the 15 residues guanylin peptide with -Leu-Lys-Lys—NH₂ was found to cause a dramatically stabilizing effect of the two topoisomers. Thus, no interconversion between the C-terminally derivatized isomers could be detected at 37° C.

Hence, in order to eliminate some of the problems with the synthesis of the guanylin we synthesized the Guanylin-(Lys)₆ (Compound 2) and the Guanylin-Leu-Lys-Lys. Further characterization of the guanylin revealed the first two amino acid residues in the N-terminal is unimportant for the biological activity of the peptide. In contrast, the tyrosine residue in position 9 is crucial for the biological activity of the peptide. Iodation of the tyrosine residue completely inactivates the peptide, however, it is not known whether the peptide is converted into an antagonist or it simply loses the ability to bind the receptor. Taking this into consideration, it is possible to make multiple guanylin peptides substituted in position 9 with tyrosine derivatives. These peptides are good antagonist candidates and can be tested in accord with methods described herein.

In vivo and in vitro efficacy assay

It has been known for nearly a decade that guanylin is an endogenous activator of intestinal guanylate cyclase as demonstrated on T84 cells, a human colon carcinoma-derived cell line. Thus, stimulation of cGMP in T84 cell cultures may be used as an efficacy assay for identifying compounds with agonistic or antagonistic guanylin receptor activity. Compound 2 was synthesized as a mixture of the two enantiomers Guanylin-(Lys)₆—NH₂ C and Guanylin-(Lys)₆—NH₂ E. The mixture and one of the enantiomers was tested in the guanylin efficacy assay described above in Example 1.

In brief, the T84 cells were grown to confluence in microtiter wells and 48-multi wells. Cells were exposed to increasing concentrations (0.1; 1.0, 10, 100, 1000 nM) of guanylin-K6 for 20 min. In an alternative experiment, cells were exposed to 10 nM guanylin-K6 for 1, 2, 5, 10, 20, & 30 min. All determinations were done in triplicate and corrected for protein content/well.

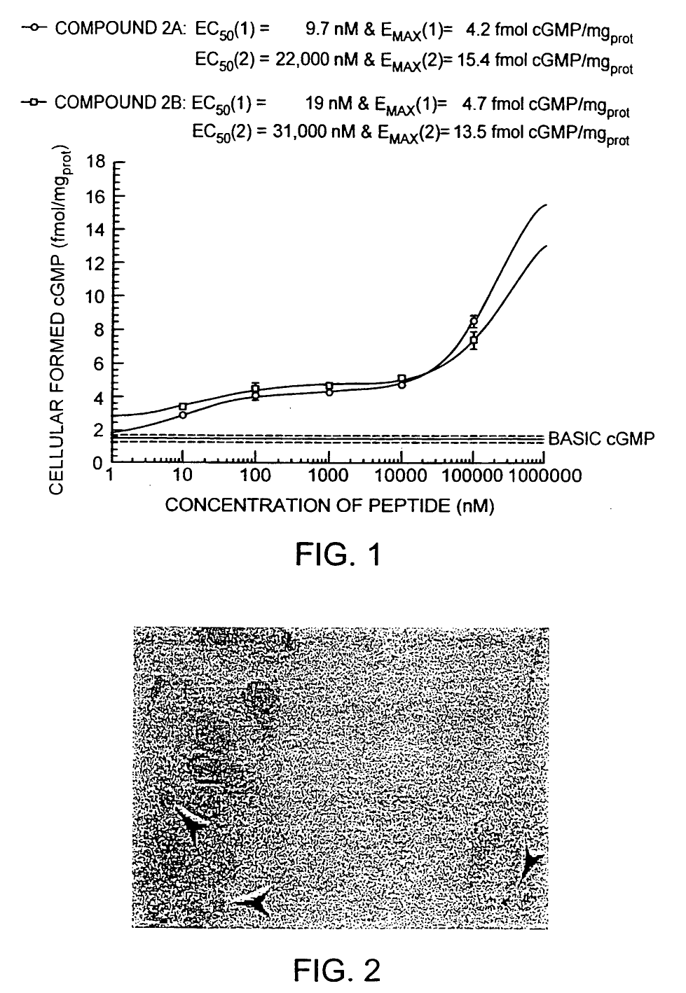

Guanylin-(Lys)₆—NH₂ C and its enantiomer Guanylin-(Lys)₆—NH₂ E behaves as very weak partial agonists (FIG. 1 ) giving rise to a 3-6 fold increase in cGMP production as compared to the 300 fold increase reported for synthetic uroguanylin and guanylin. Thus it can be concluded that it is possible to measure even very weak effects on cGMP production in T84 cells. A more potent agonist is, however, required in order to validate the efficacy assay properly. The Guanylin-(Lys)₆—NH₂ C and Guanylin-(Lys)₆—NH₂ E was also tested in vivo after iv infusion in rats to determine whether the compounds could elicit a diuretic effect. Neither Guanylin-(Lys)₆—NH₂ C nor Guanylin-(Lys)₆—NH₂ E were able to give a diuretic response in the animals at the doses used possibly due to low purity of the peptides in the preparation used. However, as can be seen in the following Example 4, a diuretic response was observed when much higher doses (bolus) of a related compound (Guanylin-Asp—OH Compound 13) was given to animals. Taken together, these results show that diuresis can be observed at relatively high doses ie., at least about 10 micromolar peptide/kilogram body weight.

Saccin and Identification of a GC-C Agonist (Compound 12)

All guanylin analogues synthesized for in vitro efficacy on the guanylate cyclase have been tested. Crude extracts from frozen saccus endolymphaticus glands have also been tested. In these experiments we could not detect any activity in the T84 assay. However, this may not be surprising because previous in vivo experiments with crude extract from frozen saccus endolymphaticus have shown that freezing completely abolishes the diuretic activity. Table 2, below shows results of selected compounds in the efficacy assay described in Example 1.

TABLE 2

| Compound | EC₅₀ μM |
|---|---|
| 1 | 6.1 |
| 3 | No effect |
| 4 | No effect |
| 5 | 28 |
| 6 | >100 |
| 7 | No effect |
| 8 | 29 |
| 9 | 59 |
| 10 | >100 |

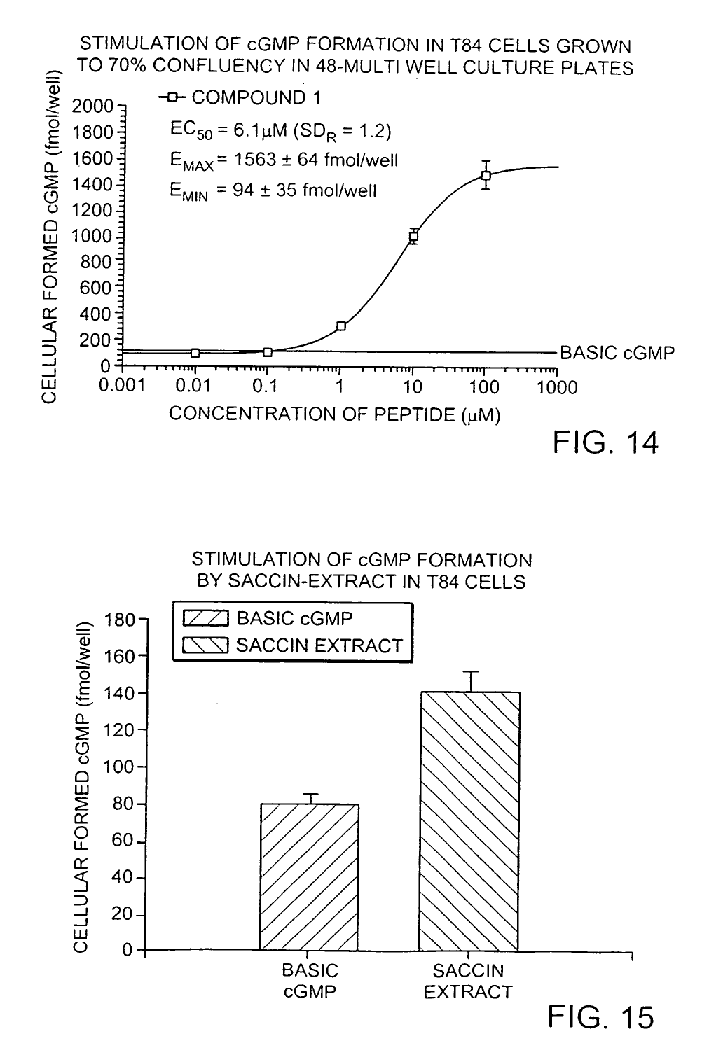

The data shown in Table 2 clearly shows that rat guanylin—NH₂ (Compound 1) is the most potent of the tested compounds. FIG. 14 confirms this observation. FIG. 14 is explained as follows: The graph shows stimulation of cGMP formation in T84 cells grown to 70% confluency in 48-multi well culture plates.

FIG. 15 shows results of stimulation of cGMP formation by saccin-extract in T84 cells. The experiment was performed along lines outlined above in Example 1.

The in vitro efficacy assay performed above in Example 1 was repeated with Compound 12. Results show stimulation of cGMP formation in T84 cells grown to 70% confluency in 48 multi-well culture plates. The results also show that compound 12 is a potent agonist of GC-C function.

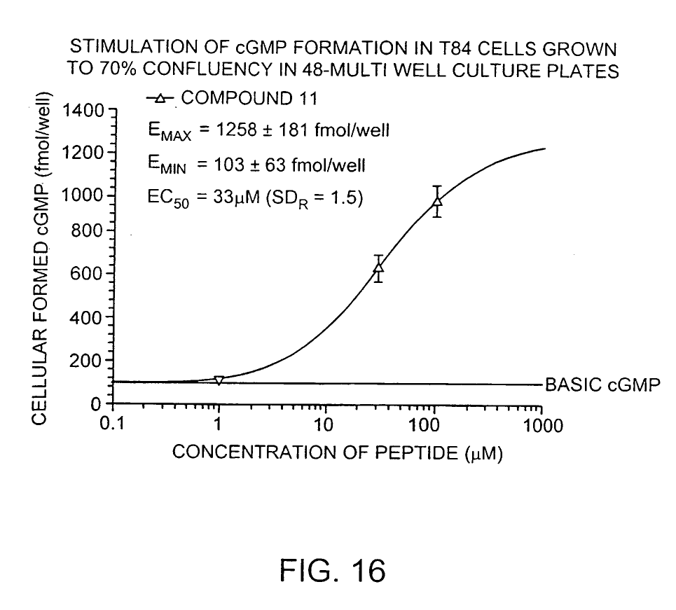

See also FIG. 16 (showing efficacy data for Compound 11).

### EXAMPLE 3

### Localization Of Guanylin and Guanylin Receptor In the Inner Ear

As discussed previously, the inner ear is filled with two types of fluid: endolymph and perilymph. These fluids bathe the inner ear's inner and outer hair cells. Because the two fluids have different electrical properties, they create an electrical charge surrounding these sensory cells. Ménière's disease results from endolymphatic hydrops which is characterized by exessive fluid within the inner ear.

There is recognition that endolymph is continually being absorbed, replenished, and cleansed. It is produced in the part of the inner ear called the stria vascularis. This apparatus works in a very similar fashion to certain portions of the kidney. This helps to explain the effectiveness of diuretics for many people with Ménière's disease. The amount of fluid that the stria produces depends on many factors, but is mainly dependent on dietary salt intake. Excess endolymph is absorbed by an organ called the endolymphatic sac. This sac lies next to the brain lining and is connected to the inner ear via a long tube.

As discussed above, it was demonstrated that matrix colloid of the endolymphatic sac had an extensive diuretic effect on rat, when administrated intravenously. The active substance in this colloid has been called saccin. Due to the diuretic effect of saccin and the localization of the saccus endolymphaticus, which is directly connected to vena aqueductus vestibularis and adjacent to sinus sigmoideus, the saccus might provide a rapid release of saccin into the blood circulation. It is believed that saccin provides a molecular basis for understanding Ménière's disease.

The present example was conducted to localize the rat guanylin and the guanylin receptor in the inner ear of the rat and to substantiate previous results showing that not only the guanylin mRNA but also the guanylin receptor mRNA could be detected in the rat inner ear.

Guanylin ligand Identification

A polyclonal antibody was raised in rabbits according to standard methods. See generally Harlow and Lane in, Antibodies: A Laboratory Manual (1988). Guanylin-K₆ (Guanylin-(Lys)₆—NH₂) was used as immunogen. After the first bleeding of the rabbits the antibody have been tested for immunoreactivity and purified. This antibody was used in all the localization studies.

Next, saccus endolymphaticus was isolated and embedded in paraffin according to standard methods. Thin sections were cut and incubated with the polyclonal antibody. The localization of guanylin was visualized by peroxidase staining.

FIG. 2 is a micrograph showing localization of guanylin to the saccus endolymphaticus (arrows).

GC-C Receptor Identification

To localize the guanylin receptor, 1 mg of COMPOUND 1 rat Guanylin—NH₂ (Compound 1) was ¹²⁵I radio labelled (Amersham Biosciences) and administrated iv. to rats. After the injection the rats were incubated with the radio labelled COMPOUND 1 Compound 1 for 5 min and the rats were subjected perfusion fixation. The stria vascularis was removed and embedded in paraffin and thin sections were cut and exposed on X-ray films for a minimum of 30 days. The films were developed and the guanylin receptor was detected as dark spots on the x-ray film.

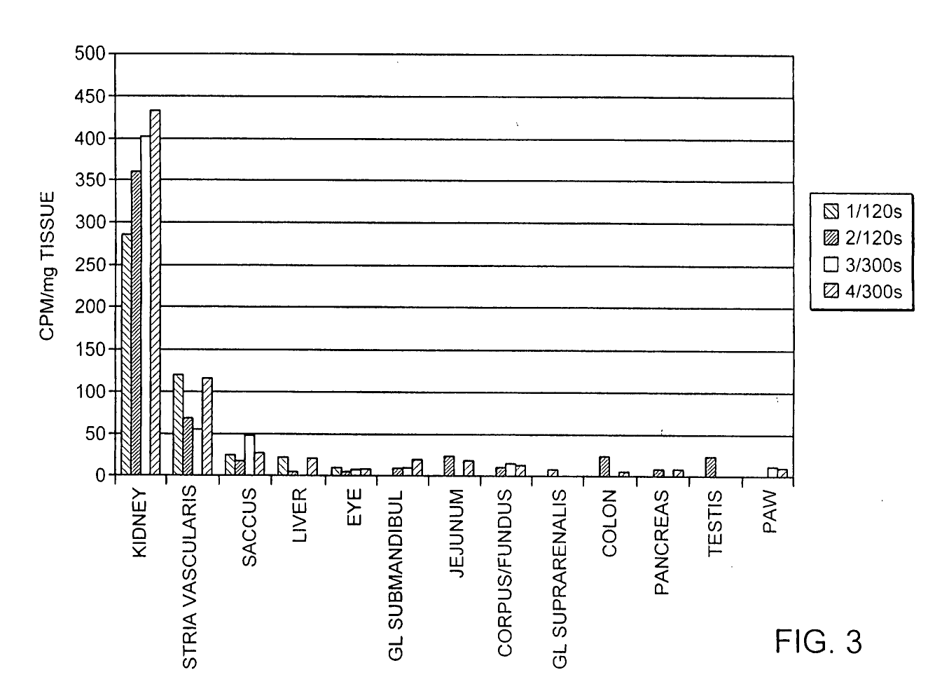

FIG. 3 is a graph showing guanylin receptor tracing in selected tissues. The results show significant presence of the GC-C receptor in kidney, stria vascularis, and the saccus.

This example shows that the mRNA encoding rat guanylin could be detected in the rat saccus endolymphaticus and that the receptor was present in the stria vascularis. To demonstrate that the guanylin protein is present and produced in the saccus endolymphaticus, polyclonals have been generated that bind rat guanylin-K6. Results show that antibody is able to recognize guanylin in the epithelial cells of the saccus endolymphaticus and in the dark luminal colloid. The data clearly show that guanylin is synthesized in the epithelial cells of the saccus endolymphaticus and transported to the lumen of the gland analogueous to the transport of T3 in the thyroid gland.

### EXAMPLE 4

### Detection Of Diuresis In The Rat

The following experiements were conducted to show that preferred compounds according to the invention exhibit significant diuretic activity. Methods for measuring diuresis and detecting sodium (natriuresis) and potassium excretion (kaliuresis) are disclosed.

Animal surgery

Male Sprague-Dawley rats weighing 350-370 g (M&B, Denmark) were anaesthetized with a subcutaneous injection of 2 ml/kg of a 1:1 mixture of Hypnorm® (fentanyl citrate 0.315 mg/ml and fluanisone 10 mg/ml) and Dormicum® (midazolam 5 mg/ml) diluted 1:1 in distilled water. Permanent catheters were inserted into the femoral vein and artery. The i.v. line is used for i.v. infusion and the arterial line is used for continuous intra-arterial blood pressure recording and withdrawal of blood samples. A permanent supra-pubic catheter was inserted into the urinary bladder, which allowed fractionated urine collection and determination of urine flow rate. Animals were allowed about one week of recovery before they were used for experiments.

Experimental procedure

On the day of the experiment, the rat was placed in a lucid restrainer. Renal clearance experiments were performed during the inactive period of the rat (i.e., daytime), when sodium- and water retaining mechanisms were maximally activated. To obtain a stable urine production under these conditions, the rat was slightly water loaded by i.v. infusion of a hypotonic glucose solution (150 mM glucose; 100 μl/min the first 15 min, then 25 μl/min throughout the experiment). The arterial line was connected to a pressure transducer (Baxter Truware: PX-260 Uden, Netherlands) and the pulsatile arterial blood pressure was amplified (×1.000-10.000) via a Hugo Sachs Electronic Tranducer Amplifier Module model 705/1. The analogue signal was digitalized via a 12 bit data acquisition board (Data Translation model DT321) and sampled at 1000 Hz using the Notocord HEM version 4 software for Windows NT. The systolic peak pressure triggered a software heart rate module that calculated heart rate beat-to-beat.

After a 60 minutes equilibration period, fractionated collection of urine was started (t=0). After 4 control periods (4×15 minutes), a bolus injection of Mouse-Guanylin-Asp—OH, 10 μmol/kg i.v was administered and collection of urine was collected in 15 minute periods for 3 hours (period 5-18). Urine volume was measured gravimetrically. The urine samples were stored at −20° C. until further analysis. Urine was analysed for sodium and potassium using a using a flame spectrophotometer (Instrumentation Laboratory, model IL 943).

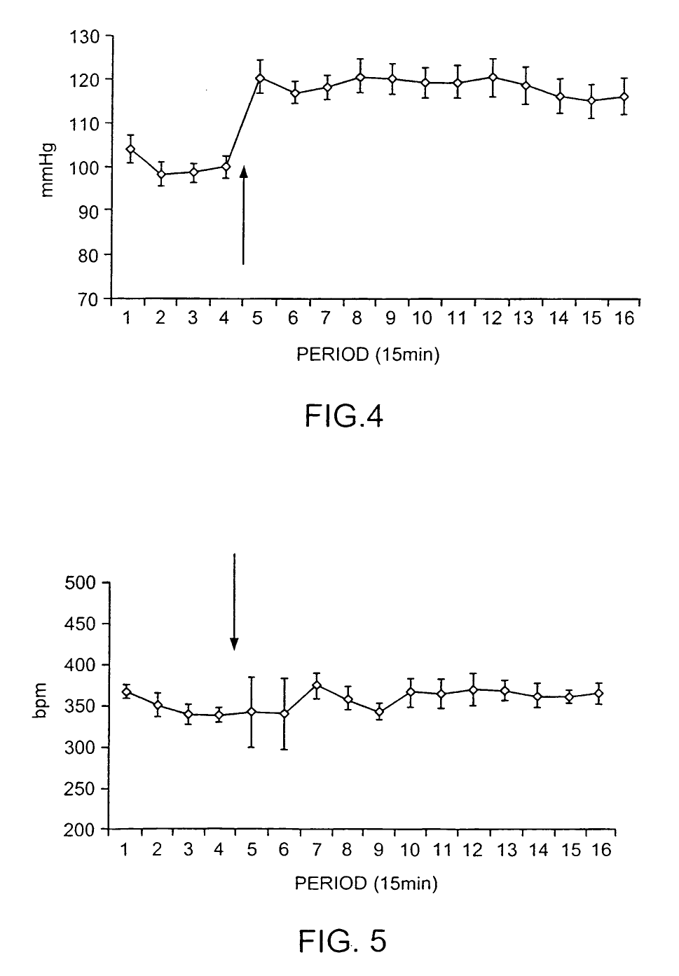

Results are presented in FIGS. 4-9. The figures are explained in more detail as follows.

FIG. 4 is a graph showing effect of Mouse-Guanylin-Asp—OH (Compound 13), 10 μmol/kg i.v. on mean arterial blood pressure in conscious rats (n=4; date are mean ±SEM). Mouse-Guanylin-Asp—OH, 10 μmol/kg i.v. produced an immediate and sustained increase in blood pressure of 15-20 mmHg magnitude. Arrow inidicates time of i.v. injection of Mouse-Guanylin-Asp—OH, 10 μmol/kg i.v.

FIG. 5 is a graph showing effect of Mouse-Guanylin-Asp—OH, 10 μmol/kg i.v. on heart rate in conscious rats (n=4; date are mean ±SEM). Mouse-Guanylin-Asp—OH, 10 μmol/kg i.v. had no effect on heart rate. Arrow inidicates time of i.v. injection of Mouse-Guanylin-Asp—OH, 10 μmol/kg i.v.

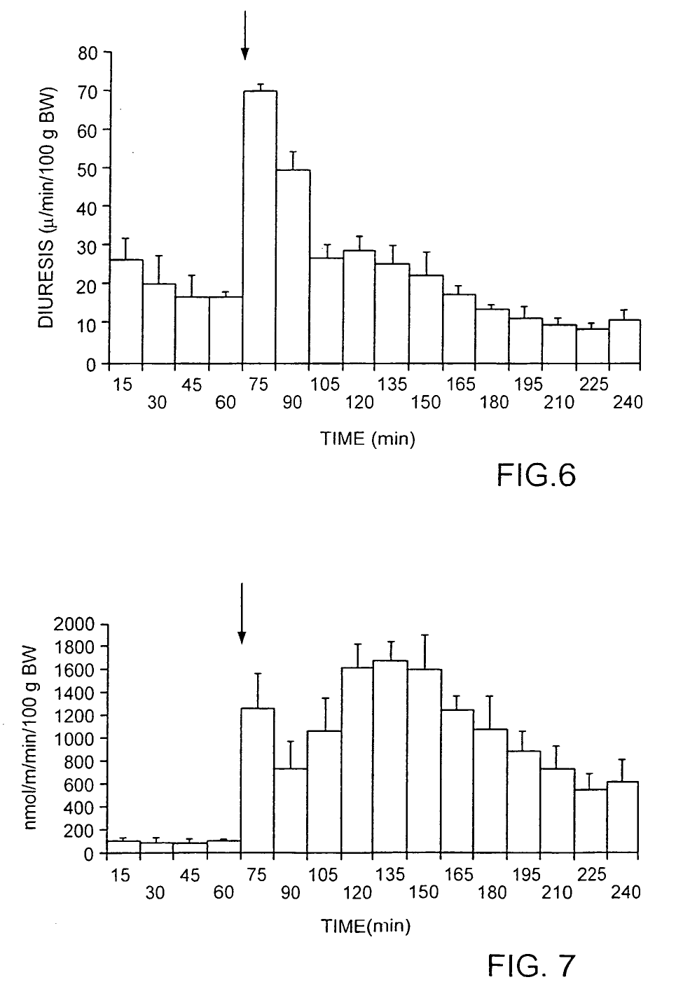

FIG. 6 is a graph showing effect of Mouse-Guanylin-Asp—OH, 10 μmol/kg i.v. on urine flow rate (n=4; date are mean ±SEM). Mouse-Guanylin-Asp—OH, 10 μmol/kg i.v. produced a rapid and transient increase in urine flow rate. Arrow inidicates time of i.v. injection of Mouse-Guanylin-Asp—OH, 10 μmol/kg i.v.

FIG. 7 is a graph showing effect of Mouse-Guanylin-Asp—OH, 10 μmol/kg i.v. on urinary sodium excretion rate (n=4; date are mean ±SEM). Mouse-Guanylin-Asp—OH, 10 μmol/kg i.v. produced an immediate and sustained increase in urinary sodium excretion rate. Arrow inidicates time of i.v. injection of Mouse-Guanylin-Asp—OH, 10 μmol/kg i.v.

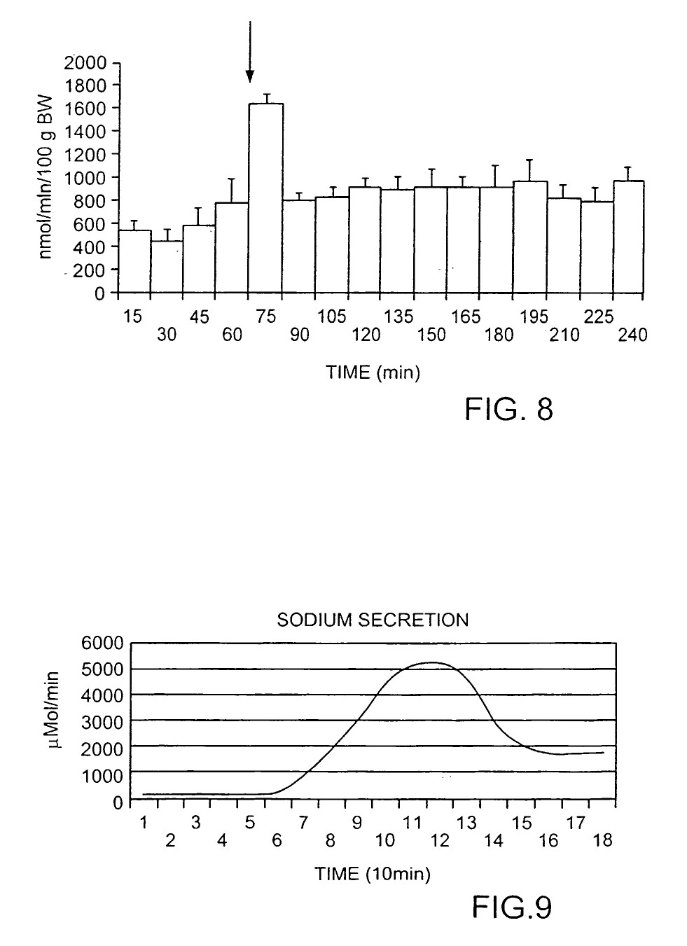

FIG. 8 is a graph showing effect of Mouse-Guanylin-Asp—OH, 10 μmol/kg i.v. on urinary potassium excretion rate (n=4; date are mean ±SEM). Mouse-Guanylin-Asp—OH, 10 μmol/kg i.v. produced a rapid and transient increase in urinary potassium excretion rate. Arrow inidicates time of i.v. injection of Mouse-Guanylin-Asp—OH, 10 μmol/kg i.v.

FIG. 9 is a graph showing sodium excretion facilitated by saccus endolymphaticus crude extract. Guanylins have nearly the same diuretic profile as the extract.

### EXAMPLE 5

### Effects of Compound 1 On Otoacoustic Emissions In Rats

The aim of the example was to determine the effect of COMPOUND 1 rat Guanylin—NH₂ on the otoacoustic emission and the renal natriuresis. That is, it is an objective of the study to determine whether guanylin exhibits the diuretic effect when administrated intraperitoneally by infusion to rat for 14 days and, most importantly, to establish whether guanylin is capable of changing the endolymph composition and thereby inducing loss of hearing, which is one of the parameters afflicted in Ménière's Disease.

In this example, rats were given 8 nmol/kg/h of Compound 1 intraperitoneally for 14 days via Alzet osmotic pumps. Otoacoustic emissions were measured before treatment and at the end of treatment. Urine samples were collected during infusion, and the natriuretic effect of Compound 1 was measured by flame spectroscopy. As shown below, the example demonstrated that 8 nmol/kg/h could induce a significant decrease in the otoacoustic emission at 14 to 16 kHz.

These results show that intraperitoneal infusion of 8 nmol/kg/h was able to induce a decrease in otoacoustic emission. Treatment of rats with agonists like COMPOUND 1 is believed to constitute a relevant model for analysing Ménière's Disease. The model has a variety of important uses including use in screening compounds that can reduce or eliminate hearing loss associated with the disease.

Materials and Methods

COMPOUND 1 was synthesised according to methods disclosed herein Identification was performed by mass spectrometry, and the purity was determined by RP-HPLC.

Following batches were pooled:

ZP 15.97, 20x(B+C)-2-C; ZP15.97, 2ox pool-3-C; ZP 24.57, 2OX 2E; the purity were 78-80%, 86% and approx. 80%, respectively with a peptide content of 93%.

Dose Levels and Groups

18 female Sprague-Dawley rats weighing 250 g each were divided into two groups. In total, 8 control animals received vehicle and 10 animals received COMPOUND 1. During the experiment the animals had free access to food and water.

Prior to the study COMPOUND 1 was dissolved in 50 mM phosphate buffer pH 4.0 containing 1% BSA and loaded into Alzet osmotic pumps. The pumps were then surgically inserted into the peritoneum of the rats administering 8 nmol/kg/h for 14 days. After insertion of the pumps the animals were placed in metabolic cages for a week, and samples of urine were collected every 24 hrs from Day 0 through Day 6.

Sodium Measurements

The collected urine samples were all analysed on an Instrumentation Laboratory 943 flame photometer.

Hearing test

The rats were all tested before the start of the study and at the end of the study. All auditory measurements were made with the same stimulus source, an Etymotic Research ER-10B+ low noise microphone system coupled to ER-2 tube phones by standard front tubes. In addition, during the tests the animals were lying on their side on a heating plate in a closed sound booth (80×80×60 cm) lined with sound absorbing material (Rockfon^{R}).

The microphone probes, which are small brass funnel fixed to a micromanipulator, were placed in the external ear. Before inserting the microphone, the position of the funnel was adjusted to allow a direct view of the eardrum with an otoscope. The primary tone frequencies f1 and f2 for the distortion product otoacoustic emission (DPOAE) were generated by an HP 8904 two-channel tone generator with phase control. The level of the stimulus from the individual ER-2 tube phones were adjusted in accordance with the frequency response curves attained from the average response (84±1.5 dBₗᵢₙ SPLᵣₘₛ) from 1 to 17.5 kHz by an ER-7C probe tube microphone at the eardrum of the animals. The output of the ER-10B and ER-7C microphones were adjusted in accordance with the frequency response curves supplied by the manufacturer.

The only DPOAE measured were the amplitude of cubic distortion product 2f1-f2, and always with the level of f1 (L1) 10 dB higher than the level of f2 (L2=L1-10 dB). The output of the ER-10B microphone was feed to a B&K (Nexus) amplifier and analysed on an HP 35670A spectrum. All test procedures and equipment were controlled by a computer and programmed in the visual programming language HP VEE (version 5.0).

DP-gram

DP-gram was obtained by measuring the CDP with a fixed ratio of f2/f1=16/13=1.23 and a fixed level of the primary tones (L2=50 dBₗᵢₙ SPLᵣₘₛ) but with varying f2 from 4096 to 17408 Hz in steps of 512 Hz. Each spectrum was made on 64 time-averaged recordings (time averaging). The DP-gram was made during anaesthesia (Hypnorm®/Dormicum®i.m.).

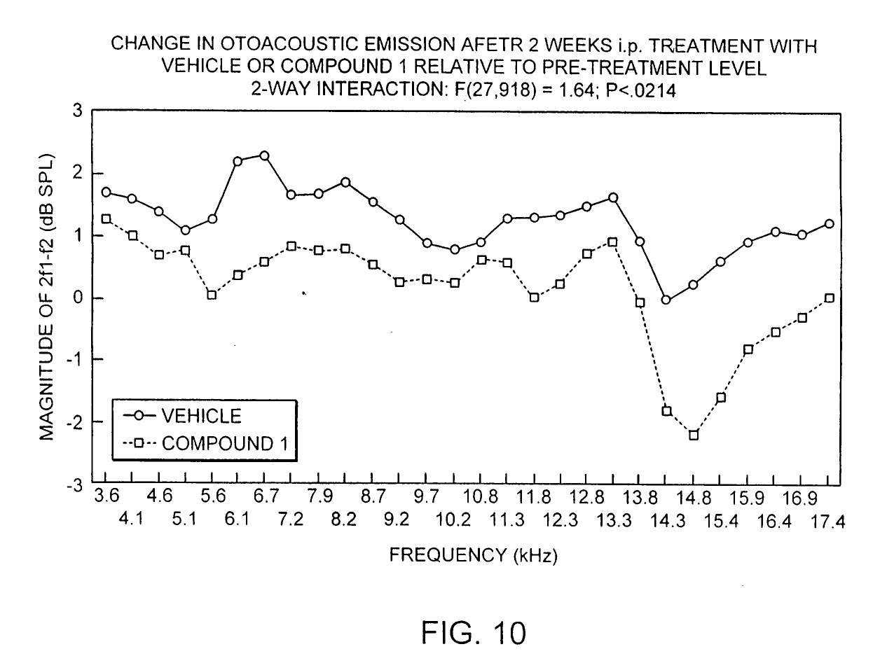

The change in otoacoustic emission from before to after treatment is shown in FIG. 10. The 2-way ANOVA analysis with repeated measures with the right and the left ear considered as one observation showed a significant decrease in the otoacoustic emission after administration of COMPOUND 1. The most pronounced decrease in the otoacoustic emission was observed at the 14 kHz level, although it seemed that COMPOUND 1 introduced a general decrease in otoacoustic emissions. Thus, administration of COMPOUND 1 for 14 days did produce a significant decrease in the otoacoustic emission, especially at the 14 kHz level.

Normal individuals have a hearing threshold from 20 Hz to 20 kHz compared with rats that have a hearing threshold from 800 Hz to 60 kHz with a maximal hearing sensitivity from 8 kHz to 30 kHz [6 and 7]. Patients suffering from Ménière's Disease normally develop a low frequency hearing loss. Since the hearing spectrum in rats is much broader than humans, we believe that the hearing loss we observe in the rats might be representative for the hearing loss observed in Ménière's Disease. In addition, covering the most sensitive spectrum from 8 kHz to 30 kHz will be of extreme importance, as the hearing loss induced might not only be restricted to the levels at 14 kHZ.

In the present example, we demonstrated that the infusion of 8 nmol/kg/h of Compound 1 elicits a reduction in hearing at 14 kHz. The results indicate that the rat model reflects hearing loss seen in patients suffering from Ménière's Disease. The model has many uses including use to detect agonists and antagonists. Antagonist compounds will be useful as therapeutics to treat symptoms of Ménière's Disease.

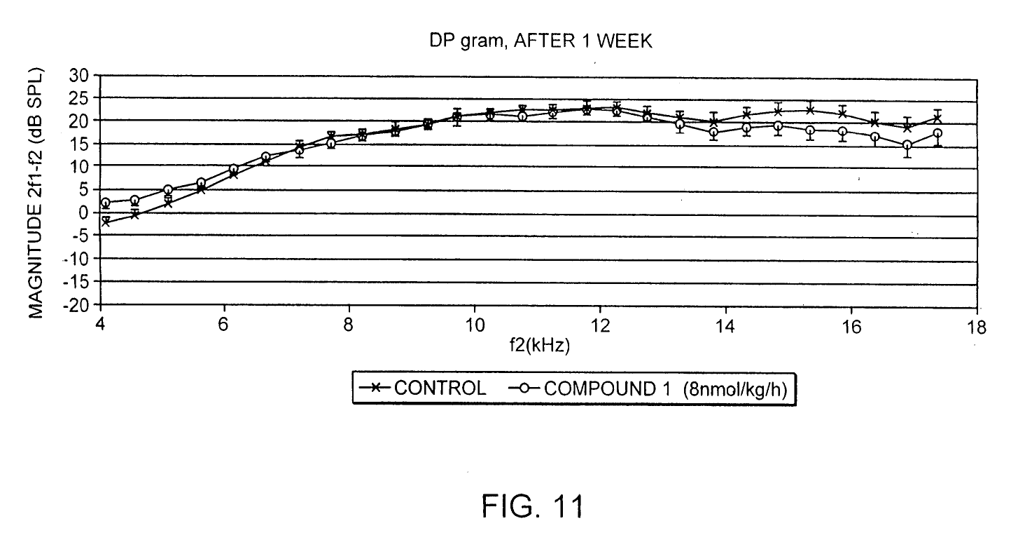

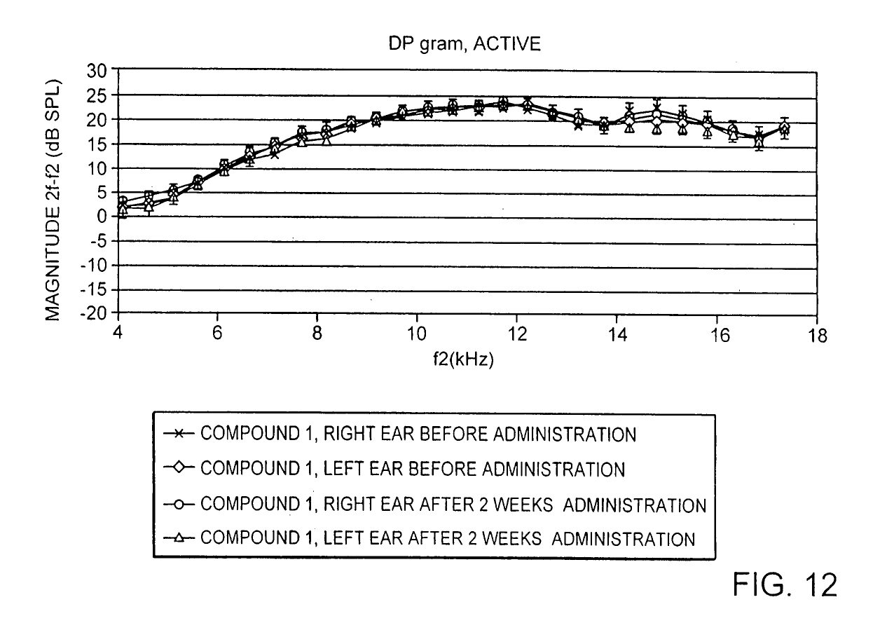

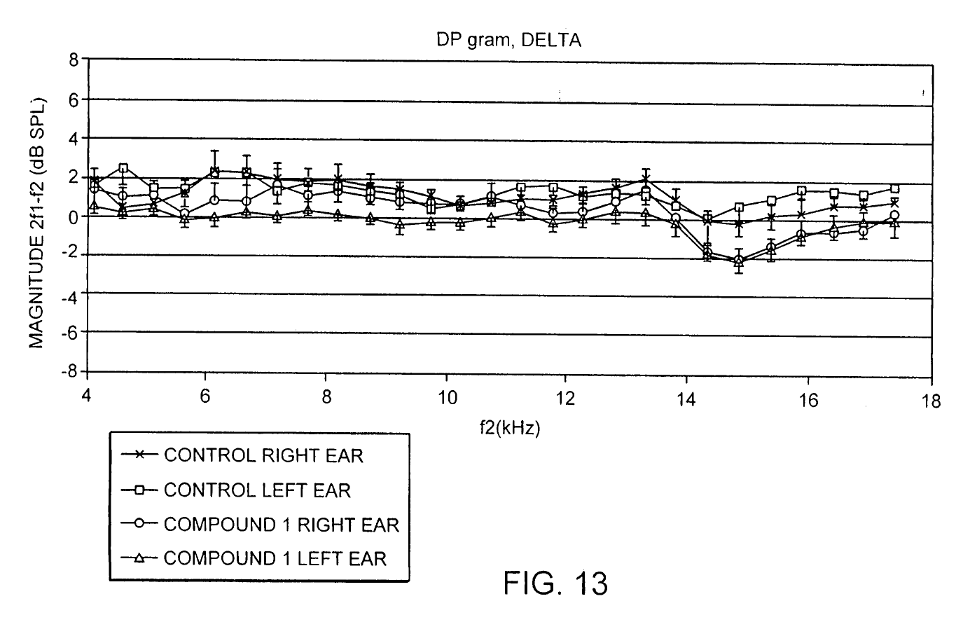

The following figures show results of the assay performed in this Example. In particular, FIG. 10 shows change in otoacoustic emission after 2 weeks i.p. treatment with vehicle or Compound 1 relative to pretreatment level. Calculation of the data from the fourteen day study showed that animals only received 8 nmol/kg/h COMPOUND 1 which correspond to a royal dose of 1.6 nmol/animal. Further 2-way ANOVA analysis with repeated measures and with the right and the left ear considered as one observation clearly revealed a significant decrease in the otoacoustic emission after administration of COMPOUND 1. FIG. 11 is a graph showing a DP-gram after 1 week; FIG. 12 is a graph showing a DP-gram, active; and FIG. 13 is a graph showing a DP-gram, delta.

The following references are referred to by number in this Example.

[1] Krause, G., Meyer, Z., Wolfram, G. and Gerzer, R.: Transcripts encoding three types of guanylyl-cyclase-coupled trans-membrane receptors in inner ear tissues of guinea pigs. Hear Res, 110, 95-106 (1997).

[2] Currie, M. G., Fok, K. F., Kato, J., Moore, R. J., Hamra, F. K., Duffin, K. L. and Smith, C. E.: Guanylin: an endogenous activator of intestinal guanylate cyclase. Proc Natl Acad Sci U S A, 89, 947-951(1992).

[3] Fonteles, M. C., Greenberg, R. N., Monteiro, H. S., Currie, M. G. and Forte, L. R.: Natriuretic and kaliuretic activities of guanylin and uroguanylin in the isolated perfused rat kidney. Am J Physiol, 275, F191-F197 (1998).

[4] Forte, L. R., London, R. M., Freeman, R. H. and Krause, W. J.: Guanylin peptides: renal actions mediated by cyclic GMP. Am J Physiol Renal Physiol, 278, F180-F191 (2000).

[5] Qvortrup, K., Rostgaard, J. and Holstein-Rathlou, N.-H.: The inner ear produces a natriuretic hormone. Am J Physiol, 270, F1073-F1077 (1996).

[6] Syka, J and Rybalko, N.: Threshold shifts and enhancement of cortical evoked responses after noise exposure in rats. Hear Res, 139, 59-68 (2000).

[7] Fausti, S. A., Erickson, D. A., Frey, R. H., Rappaport, B. Z. and Schechter, M. A.: The effects of noise upon human hearing sensitivity from 8000 to 20 000 Hz. J Acoust Soc Am, 69, 1343-1347 (1981). See also Schulz, S., Green,C. K., Yuen, P. S, and Garbers.D. L.(1990). Guanylyl Cyclase Is a Heat-stable Enterotoxin Receptor.Cell, 63, 941-948,

### EXAMPLE 6

### Synthesis of authentic rat guanylin

A small batch of authentic rat guanylin was made for efficacy measurements (see Example 1, above). Furthermore, production of batches of proguanylin was also initiated. Former results have shown that synthesis of guanylin is facilitated by a C terminal SIP modification. A SIP (Structure Inducing Probe) is a short peptide sequence consisting of alpha amino acid residues selected from the group of amino acids having desired values of propensity factors selected from propensity factor Pα>0.57 and a propensity factor Pβ≦1.10, cf. WO 98/11125 and Chou and Fasman, 1978, Ann. Rev. Biochem. 47:251-276, the description of which is incorporated herein by reference. After synthesis, the SIP modification is removed by enzymatical digestion with suitable endopeptidases gaining authentic guanylin. However, pilot experiments with enzymatic digestion have shown that enzyme digestion is not complete giving rise to C-terminally extended guanylin analogues, such as guanylin-DK. This guanylin analogue can be tested in the T84 efficacy assay disclosed above in Example 1. Synthesis of Guanylin-MK₅ for solid phase enzymatic cleavage with CNBr is another possibility.

### EXAMPLE 7

### Immunogen Production

Antibodies specific for Compound 11 will be raised against Compound 11 which is a peptide epitope derived from the GC-C sequence (Hasegawa, M. et al. The Journal of Biological Chemistry. 1999. Vol. 274 (44), pp-31713-31718) having an extra terminal Cys residue to accomodate the coupling chemistry. H-SPNFIWKC—NH₂ will be coupled to keyhole limpet hemocyanin (KLH) and a commercially available cationised bovine serum albumin (Supercarrier, Pierce Chemical). The resulting KLH and Supercarrier conjugates were combined, and injected into rabbits. Compound 11 was conjugated to purified protein derivative (PPD) and injected into a BCG primed goat. Immunisations followed standard protocols of initial immunisation of immunogen in an emulsion of Freund's complete adjuvant followed by a regimen of boosting with an emulsion of the immunogen in Freund's incomplete adjuvant and test bleeding until antibody titers were at acceptable levels. The antibodies were purified in one step by affinity chromatography on a protein G column specific for the IgG subclass of immunoglobulins.

### EXAMPLE 8

### Antibody Production

Antibodies were prepared generally along lines discussed previously. Three representative polyclonal antibodies will be raised in rabbits to peptides fitting the description in Example 7 and one representative polyclonal antibody will be raised in goat to the full sequence of Compound 11. These polyclonal antibodies are highly specific when the antibodies are evaluated in a standard ELISA type assay employing Compound 11 as the antigen and utilizing colorimetric detection of bound antibody by a commercially available HRP-conjugated anti-IgG antibody.

### EXAMPLE 9

### Synthesis of Guanylin and Guanylin Analogues

Apparatus and synthetic strategy

Peptides were synthesized batchwise in a polyethylene vessel equipped with a polypropylene filter for filtration using 9-fluorenylmethyloxycarbonyl (Fmoc) as N-α-amino protecting group and suitable common protection groups for side-chain functionalities.

Solvents

Solvent DMF (N,N-dimethylformamide, Riedel de-Häten, Germany) was purified by passing through a column packed with a strong cation exchange resin (Lewatit S 100 MB/H strong acid, Bayer AG Leverkusen, Germany) and analyzed for free amines prior to use by addition of 3,4-dihydro-3-hydroxy-4-oxo-1,2,3-benzotriazine (Dhbt—OH) giving rise to a yellow color (Dhbt-O⁻ anion) if free amines are present. Solvent DCM (dichloromethane, analytical grade, Riedel de-Häen, Germany) was used directly without purification. Chloroform, (analytical grade, Riedel de-Häen, Germany) was used directly without purification. Acetonitril (HPLC-grade, Lab-Scan, Dublin Ireland) was used directly without purification.

Amino acids

Fmoc-protected amino acids were purchased from Advanced ChemTech (ACT) in suitabel side-chain protected forms. Otherwise protected amino acids Fmoc-Glu(OH)-OAllyl; Fmoc-Asp(OH)-OAllyl from NovaBiochem (Switzerland).

Coupling reagents

Coupling reagent diisopropylcarbodiimide (DIC) was purchased from (Riedel de-Häen, Germany), PyBop from Advanced ChemTech.

Solid supports

Peptides were synthesized on TentaGel S resins 0,23-0,24 mmol/g. TentaGel S-Ram, TentaGel S RAM-Lys(Boc)Fmoc (Rapp polymere, Germany).

Catalysts and other reagents

Diisopropylethylamine (DIEA) was purchased from Aldrich, Germany, piperidine and pyridine from Riedel-de Häen, Frankfurt, Germany. Ethandithiol was purchased from Riedel-de Häen, Frankfurt, Germany. 3,4-dihydro-3-hydroxy4-oxo-1,2,3-benzotriazine (Dhbt—OH), 1-hydroxybenzotriazole (HOBt) (HOAt) were obtained from Fluka, Switzerland.

Coupling procedures

The amino acids were coupled as in situ generated HObt or HOAt esters made from appropriate N-a-protected amino acids and HObt or HOAt by means of DIC in DMF. Acylations were contiued for 2 h and checked by the ninhydrin test performed at 80° C. in order to prevent Fmoc deprotection during the test (Larsen, B. D. and Holm, A., Int. J. Peptide Protein Res. 43, 1994, 1-9).

Deprotection of the N-alpha-amino protecting group (Fmoc).

Deprotection of the Fmoc group was performed by treatment with 20% piperidine in DMF (1×5 and 1×10 min.), followed by wash with DMF (5 times, 5 min. each) until no yellow color could be detected after addition of Dhbt—OH to the drained DMF.

Deprotection of allyl

A solution of 3 eq. Pd(PPh₃)₄ dissolved in 15-20 ml CHCl₃, ACOH, NMM (37:2:1) was added to the peptide resin. The treatment was continued for three hours at room temperature accompanied by bubbling a stream of N₂ through the mixture.

Coupling of HOBt-esters

3 eq. N-alpha-amino protected amino acid was dissolved in DMF together with 3 eq. HObt and 3 eq. DIC and then added to the resin.

Cyclization of the peptide on the resin

1,5 eq. PyBop was dissolved in DMF together with 1,5 eq. HOBt and 3 eq. NMM was added to the peptide resin. The reaction was continued over night.

Cleavage of peptide from resin with acid.

Peptides were cleaved from the resins by treatment with 95% triflouroacetic acid (TFA, Riedel-de Häen, Frankfurt, Germany)-water v/v or with 95% TFA and 5% ethandithiol v/v at r.t. for 2 h. The filtered resins were washed with 95% TFA-water and filtrates and washings evaporated under reduced pressure. The residue was washed with ether and freeze dried from acetic acid-water. The crude freeze dried product was analyzed by high-performance liquid chromatography (HPLC) and identified by mass spectrometry (MS).

Batchwise peptide synthesis on TentaGel resin (PEG-PS).

TentaGel resin (0.23-0.24 mmol/g) was placed in a polyethylene vessel equipped with a polypropylene filter for filtration. The resin was swelled in DMF, and treated with 20% piperidine in DMF in order to remove the initial Fmoc group either on the linker TentaGel S RAM or on the first amino acid on the resin TentaGel S RAM-Lys(Boc)Fmoc. The resin was drained and washed with DMF until no yellow color could be detected after addition of Dhbt—OH to the drained DMF. The amino acids according to the sequence were coupled as preformed Fmoc-protected HObt esters (3 eq.) as described above. The couplings were continued for 2 h, unless otherwise specified. The resin was drained and washed with DMF (5 times, 5 min each) in order to remove excess reagent. All acylations were checked by the ninhydrin test as described above. After completed synthesis the peptide-resin was washed with DMF (3 times, 5 min each), DCM (3 times, 1 min each) and finally diethyl ether (3 times, 1 min each) and dried in vacuo. The peptide was cleaved from the resin as described earlier and the crude peptide product was analysed and purified as described below

HPLC conditions

Gradient HPLC analysis was done using a Hewlett Packard HP 1100 HPLC system consisting of a HP 1100 Quaternary Pump, a HP 1100 Autosampler a HP 1100 Column Thermostat and HP 1100 Multiple Wavelength Detector. Hewlett Packard Chemstation for LC software (rev. A.06.01) was used for instrument control and data acquisition. The following columns and HPLC buffer system was used:

Column

VYDAC 238TP5415, C-18, 5 m, 300Å 150×4.6 mm.

Buffer system

Buffers: A: 0,1% TFA in MQV; B: 0,085% TFA, 10% MQV, 90% MeCN.

Gradient:

0-1,5 min. 0% B

1,5-25 min 50% B

25-30 min 100% B

30-35 min 100% B

35-40 min 0 % B

Flow 1, ml/min, oven temperature 40° C., UV detection: λ=215 nm.

HPLC purification of the crude peptide

The crude peptide products were purified PerSeptive Biosystems VISION Workstation. VISION 3.0 software was used for instrument control and data acquisition. The following column and HPLC buffer system was used:

Column

Kromasil KR 100Å, 10 m C-8, 250×50.8 mm.

Buffer system

Buffers: A: 0,1% TFA in MQV; B: 0,085% TFA, 10% MQV, 90% MeCN.

Gradient: sufficient

Flow 35 ml/min,

UV detection: λ=215 nm and 280 nm.

Mass spectroscopy

The peptides were dissolved in super gradient methanol (Labscan, Dublin, Ireland), milli-Q water (Millipore, Bedford, Mass.) and formic acid (Merck, Damstadt, Germany) (50:50:0.1 v/v/v) to give concentrations between 1 and 10 μg/ml. The peptide solutions (20 μl) were analysed in positive polarity mode by ESI-TOF-MS using a LCT mass spectrometer (Micromass, Manchester, UK).

Disulphide bond formation

A. by means of DMSO oxidation.

The purified Guanylin peptide having deprotected thiol groups at Cys7 and Cys15 is dissolved in 5% acetic acid (v/v) pH adjusted to 6 by adding sodium hydroxide(conc. 1 mg/ml). 20% by volume of DMSO was added. The progress of the oxidation was followed using analytical RP-HPLC. The oxidation reaction was allowed to proceed over night. After completion of the reaction the mixture was loaded directly into the preparative RP-HPLC column and purified as described earlier.

B. by means of air oxidation.

The purified Guanylin peptide having deprotected thiol groups at Cys7 and Cys15 is dissolved (conc. 1 mg/ml) in Ammoniumbicarbonate (0.1M) buffer pH 8. The solution is stirred vigorously at room temperature. The progress of the oxidation is followed using analytical RP-HPLC. The oxidation reaction was stopped after 10 days and the resulting mixture was lyophilised and the crude material was purified using preparative RP-HPLC as described earlier.

C. by means of iodine oxidation.

The purified oxidised Guanylin peptide Acm-protected at Cys4 and Cys12 is dissolved (1 mg/ml) in 0.1 M HCl/acetic acid 1:4 (v/v). 20 eq. Iodine dissolved in methanol (20 mg/ml) is added to the peptide solution. The reaction is left at room temperature for 24 h covered from light. The reaction volume is doubled by adding water and the excess iodine is removed by extraction using chloroform. The solution is lyophilised and the crude peptide product is purified using RP-HPLC as earlier described.

### EXAMPLE 10

### Removal of the C-terminal presequence-(Lysine)₆—NH₂ using a combination of Trypsin and Carboxypeptidase B

The Guanylin-(Lys)₆ or Guanylin-Asp-(Lys)₆ peptide is dissolved in ammoniumbicarbonate (1 mg/ml) pH 8 and preheated to 37° C. Trypsin dissolved in 0.1M HCl is added to the peptide solution (1:10 enz/peptide g/g). The reaction is left at 37° C. for 1 hour. Carboxypeptidase B dissolved in 0.1M HCl is then added to the solution (1:20 enzyme/peptide g/g) and the reaction is continued at 37° C. for another 4 hours. TFA is added until a concentration of 5% v/v in order to quench the enzymes. The solution is filtered and lyophilised and the crude peptide product is purified using RP-HPLC as earlier described.

### EXAMPLE 11

### Removal of the C-terminal Aspartic acid using Carboxypeptidase A

The Guanylin-Asp—OH (Compound 13) peptide is dissolved in ammoniumbicarbonate (1 mg/ml) pH 8 and preheated to 37° C. Carboxypeptidase A—solution is added (1:10 enzyme/peptide g/g). The reaction is left for 6 hours at 37° C. TFA is added until a concentration of 5% v/v in order to quench the enzymes. The solution is filtered and lyophilised and the crude peptide product Guanylin—OH (Compound 14) is purified using RP-HPLC as earlier described.

The presequence attached to the C-terminus in several of the Guanylin-peptides herein can be removed using endopeptidases and/or carboxypeptidases, such as Trypsin, Carboxypeptidase A and B and others. The person skilled in the art will know how to select a useful enzyme, such as a peptidase, for the removal of C-terminal amino acid residues. A general description of suitable endo- and carboxypeptidases, as well as other enzymes can be found in Sambrook et al. in Molecular Cloning: A Laboratory Manual (2d ed. 1989); Ausubel et al. (1989), Current Protocols in Molecular Biology, John Wiley & Sons, New York; as well as references cited therein.

### EXAMPLE 12

### Synthesis of Compound 12

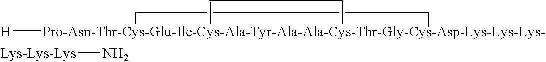

Synthesis of a.

H-Pro-Asn-Thr-Cys(Acm)-Glu-Ile-Cys-Ala-Tyr-Ala-Ala-Cys(Acm)-Thr-Gly-Cys-Asp-Lys-Lys-Lys-Lys-Lys-Lys—NH₂ on TentaGel S RAM-Lys(Boc)Fmoc. Dry TentaGel S RAM-Lys(Boc)Fmoc (0.24 mmol/g, 10 g) was placed in a polyethylene reaction vessel equipped with a polypropylene filter for filtration and treated as described under “batchwise peptide synthesis on TentaGel resin” until finishing the coupling of the N-terminal Proline. All couplings were continued for 2 h. After completed synthesis the peptide was cleaved from the resin as described above. Yield of crude product 5.86 g. After purification using preparativ HPLC as earlier described, 4.18 g peptide product was collected with a purity better than 60% and the identity of the peptide was confirmed by MS (found M 2543.11, calculated M 2543.25).

Synthesis of b.

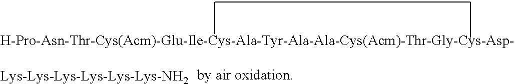

The purified peptide H-Pro-Asn-Thr-Cys(Acm)-Glu-Ile-Cys-Ala-Tyr-Ala-Ala-Cys(Acm)-Thr-Gly-Cys-Asp-Lys-Lys-Lys-Lys-Lys-Lys—NH₂ (4.18 g) was air oxidised or DMSO oxidised as described earlier. 4.1 g crude material was isolated after lyophilisation. The crude material was purified using RP-HPLC as described earlier, yielding 783.2 mg. The identity of the peptide was confirmed by MS (found M 2541.11, calculated M 2541.25).

Synthesis of c.

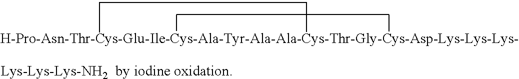

The purified peptide

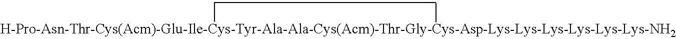

(783 mg) was oxidised by means of iodine as described earlier. The crude material was purified using RP-HPLC as described earlier, yielding 335 mg. The identity of the peptide was confirmed by MS (found M 2397.09, calculated M 2397.18).

### EXAMPLE 13

### Synthesis of Compound 3: H-Ser-Pro-Asn-Phe-Ile-Trp-Lys—NH₂

Dry TentaGel S RAM-Lys(Boc)Fmoc (0.24 mmol/g, 1 g) was placed in a polyethylene reaction vessel equipped with a polypropylene filter for filtration and treated as described under “batchwise peptide synthesis on TentaGel resin” until finishing the coupling of the N-terminal Serine. All couplings were continued for 2 h. After completed synthesis the peptide was cleaved from the resin as described above. Yield of crude product 172 mg. After purification using preparativ HPLC as earlier described, 72.6 mg peptide product was collected with a purity better than 97% and the identity of the peptide was confirmed by MS (found M 889.38, calculated M 889.48). Compound 11 was synthesised according to the same procedure, cf. Table 3 below.

### EXAMPLE 14

### Synthesis of Compound 4: Cyclo(Gly-Glu-Ile-Gly-Ala-Tyr-Ala-Ala-Gly-Thr-Gly-Gln-)

Dry TentaGel S RAM (0.23 mmol/g, 1 g) was placed in a polyethylene reaction vessel equipped with a polypropylene filter for filtration and treated as described under “batchwise peptide synthesis on TentaGel resin” until finishing the coupling of the N-terminal Glycine. All couplings were continued for 2 h. After completed synthesis the allyl protecting group on the C-terminal Glutamine was removed as described earlier and the peptide was cyclized as described earlier. The cyclo-peptide was cleaved from the resin as described above. Yield of crude product 40 mg. After purification using preparativ HPLC as earlier described, 17 mg peptide product was collected. The identity of the peptide was confirmed by MS (found M 1075.00, calculated M 1075.49).

### EXAMPLE 15

### Synthesis of Compound 13

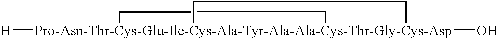

300 mg Compound 12 was treated with a combination of Trypsin and Carboxypeptidase B in order to remove the presequence (Lys)₆ from the C-terminus of the peptide as described above. After purification of the crude peptide product 69 mg of Compound 13 was isolated. The identity of the peptide was confirmed by MS (found M 1629.58, calculated M 1629.53).

### EXAMPLE 16

### Synthesis of Compound 14: Guanylin—OH

Compound 13 was treated with Carboxypeptidase A as described above. After purification using preparative RP-HPLC as described above Guanylin was isolated and the identity of the peptide was confirmed by MS (found M 1514.61, calculated M 1514.53).

TABLE 3
Summary of peptide synthetic procedures and MS data.

| | Peptide sequences. Guanylin peptides | Synthetic | MS | |
|---|---|---|---|---|
| Compound No. | are of the mouse/rat isoform | procedure | Found | Calculate |
| 1 | Guanylin-NH₂ | Acc. Ex 9 | 1513.50 | 1513.58 |
| 2 | Guanylin-(Lys)₆-NH₂ | Acc. Ex 9 | 2282.00 | 2282.15 |
| 3 | H-SPNFIWK-NH₂ | Acc. Ex 13 | 889.38 | 889.48 |
| 4 | Cyclo(GEIGAYAAGTGQ) | Acc. Ex 14 | 1075.00 | 1075.49 |
| 5 | Des-Pro¹,des-Asn²-[Phe]⁹Guanylin-Leu- | Acc. Ex 9 | 1399.63 | 1399.57 |
| | NH₂ | | | |
| 6 | Des-Pro¹,des-Asn²- | Acc. Ex 9 | 1449.63 | 1449.59 |
| | [2-Nal]⁹Guanylin-Leu-NH₂ | | | |
| 7 | Des-Pro¹,des-Asn²-Guanylin-Leu-(Lys)₂- | Acc. Ex 9 | 1815.88 | 1815.85 |
| | NH₂ (oxidised Cys⁷-Cys¹⁵) | | | |
| 8 | Des-Pro¹,des-Asn²-Guanylin-Leu-(Lys)₂- | Acc. Ex 9* | 1671.77 | 1671.76 |
| | NH₂ (oxidised Cys⁴-Cys¹² and Cys⁷-Cys¹⁵) | | | |
| | (single topo-isomer) | | | |
| 9 | Des-Pro¹,des-Asn²-Guanylin-Leu-(Lys)₂- | ACC. EX 9 | 1671.77 | 1671.76 |
| | NH₂ (oxidised Cys⁴-Cys¹² and Cys⁷-Cys¹⁵) | | | |
| | (mixture of topo-isomers) | | | |
| 10 | Des-Pro¹,des-Asn²-Guanylin-Leu-(Lys)₂- | Acc. Ex 9* | 1671.77 | 1671.76 |
| | NH₂ (oxidised Cys⁴-Cys¹² and Cys⁷-Cys¹⁵) | | | |
| | (single topo-isomer) | | | |
| 11 | H-SPNFIWKC-NH₂ | Acc. Ex 9 | 992.37 | 992.49 |
| 12 | Guanylin-Asp-(Lys)₆-NH₂ | Acc. Ex 9 | 2541.25 | 2541.18 |
| 13 | Guanylin-Asp-OH | Acc. Ex 10 | 1629.58 | 1629.53 |
| 14 | Guanylin-OH | Acc. Ex 11 | 1514.61 | 1514.53 |

*The topo-isomers were separated using preparative RP-HPLC as earlier described.

What is claimed is:

1. A cyclic polypeptide, that is capable of inhibiting binding of Guanylin or a compound which comprises Guanylin like activity to Guanylyl Cyclase C.

2. The cyclic polypeptide according to claim 1, wherein the said cyclic polypeptide has the amino acid sequence: 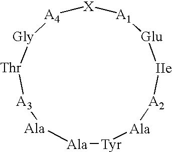
wherein A₁, A₂, A₃, and A₄ could be any amino acid and X is a bond or a spacer.

3. The cyclic polypeptide according to claim 1, wherein the cyclic polypeptide has the amino acid sequence: 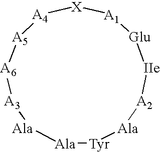
wherein A₁, A₂, A₃, A₄, A₅, and A₆ could be any amino acid and X is a bond or a spacer.

4. The cyclic polypeptide according to claim 3, wherein A₅ is selected from the group consisting of Ser and Ala.

5. The cyclic polypeptide according to claim 3, wherein A₆ is selected from the group consisting of Ser and Ala.

6. The cyclic polypeptide according to claim 1, wherein the cyclic polypeptide has the amino acid sequence: 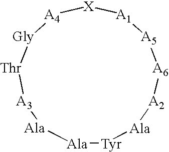
wherein A₁, A₂, A₃, A₄, A₅, and A₆ could be any amino acid and X is a bond or a spacer.

7. The cyclic polypeptide according to claim 6, wherein A₅ is selected from the group consisting of Asp, Asn, Gln, Leu, Val, Ala and Gly.

8. The cyclic polypeptide according to claim 6, wherein A₆ is selected from the group consisting of Asp, Asn, Gln, Leu, Val, Ala and Gly.

9. The cyclic polypeptide according to claim 1, wherein the cyclic polypeptide has the amino acid sequence 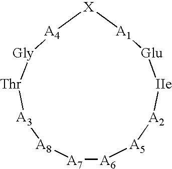
wherein A1-A8 could be any amino acid and X is a bond or a spacer.

10. The cyclic polypeptide according to claim 9, wherein A₅ is selected from the group consisting of Ala, Gly, Val, Leu, Ile, Asn, Gln, Tyr, His and Trp.

11. The cyclic polypeptide according to claim 9, wherein A₆ is selected from the group consisting of Ala, Gly, Val, Leu, Ile, Asn, Gln, Tyr, His and Trp.

12. The cyclic polypeptide according to claim 9, wherein A₇ is selected from the group consisting of Ala, Gly, Val, Leu, Ile, Asn, Gln, Tyr, His and Trp.

13. The cyclic polypeptide according to claim 9, wherein A₈ is selected from the group consisting of Ala, Gly, Val, Leu, Ile, Asn, Gln, Tyr, His and Trp.

14. The cyclic polypeptide according to any of the claims 2-13, wherein A₁ is selected from the group consisting of Gly, Cys, Ser, Lys, Lysine analogues, Thr, Glu and Ala.

15. The cyclic polypeptide according to any of the claims 2-14, wherein A₂ is selected from the group consisting of Gly and Ala.

16. The cyclic polypeptide according to any of the claims 2-15, wherein A₃ is selected from the group consisting of Gly and Ala.

17. The cyclic polypeptide according to any of the claims 2-16, wherein A₄ is selected from the group consisting of Gly, Cys, Ser, Lys, Lysine analogues, Thr, Glu and Ala.

18. An antibody that specifically binds to a Guanylyl Cyclase C polypeptide which comprises an epitope comprising the amino acid sequence Pro-Xaa-Phe-Xaa-Trp, and wherein said antibody is capable of binding said epitope.

19. An antibody that specifically binds to a Guanylyl Cyclase C epitope and wherein said epitope comprise the amino acid sequence Xaa-Pro-Xaa-Phe-Xaa-Trp.

20. The antibody according to any of the claims 18-19, wherein said epitope comprises an amino acid sequence selected from the group consisting of: a) Ser-Pro-Thr-Phe-Ile-Trp-Lys; b) Ser-Pro-Thr-Phe-Thr-Trp-Lys; c) His-Pro-Thr-Phe-Thr-Trp-Lys; d) Ser-Pro-Asn-Phe-Ile-Trp-Lys; e) Ser-Pro-Thr-Phe-Ile-Trp; f)H-SPNFIWK—NH₂; g) H-SPNFITK—NH₂; H-SPNFITKC—NH₂; and H-SPNFIWKC—NH₂.

21. The antibody according to any of the claims 18-20, wherein said antibody is a monoclonal antibody.

22. The antibody according to any of the claim 18-21, wherein said antibody has been isolated.

23. The antibody according to any of the claims 18-22, wherein said antibody is humanised.

24. The antibody according to any of the claims 18-23, wherein said antibody is a polyclonal antibody.

25. The antibody according to any of the claims 18-24, wherein said antibody further comprises a labelling group selected from the group consisting of: a fluorescent label, a radioactive label, a bioactive label and a heavy metal.

26. Use of an inhibitor of guanylyl cyclase C activity for the preparation of a medicament for the treatment of Ménière's disease or the symptoms tinnitus, vertigo and hearing loss related to Ménière's disease.

27. Use according to claim 26 wherein said inhibitor is selected from the group consisting of methylene blue; 1H-[1,2,4]oxadiazolo[4,3-a]quinoxalin-1-one (ODQ); 6-(phenylamino)-5,8-quinolinedione; guanosine 5′-tetraphosphate; adenosine 5′-tetraphosphate and Rp-GTPalphaS.

28. A pharmaceutical composition comprising a therapeutically effective amount of an inhibitor, wherein said inhibitor is capable of inhibiting binding of Guanylin or a compound which comprises Guanylin like activity to Guanylyl Cyclase C, together with a pharmaceutically acceptable carrier.

29. The pharmaceutical composition according to claim 28, wherein the inhibitor is capable of associating with Guanylin.

30. The pharmaceutical composition according to claim 28 or 29, wherein the inhibitor is a polypeptide.

31. The pharmaceutical composition according to claim 30, wherein the inhibitor is a polypeptide comprising approximately 15 amino acids.

32. The pharmaceutical composition according to claim 30, wherein the inhibitor is a heptapeptide.

33. The pharmaceutical composition according to claim 30, wherein the inhibitor is a hexapeptide.

34. The pharmaceutical composition according to claim 30, wherein the inhibitor is a pentapeptide.

35. The pharmaceutical composition according to claim 30, wherein the inhibitor is a polypeptide comprising the amino acid sequence Xaa-Pro-Xaa-Phe-Xaa-Trp.

36. The pharmaceutical composition according to claim 30, wherein the inhibitor is a polypeptide comprising an amino acid sequence selected from the group consisting of: a) Ser-Pro-Thr-Phe-Ile-Trp-Lys; b) Ser-Pro-Thr-Phe-Thr-Trp-Lys; c) His-Pro-Thr-Phe-Thr-Trp-Lys; d) Ser-Pro-Asn-Phe-Ile-Trp-Lys; and e) Ser-Pro-Thr-Phe-Ile-Trp; f)H-SPNFIWK—NH₂; g) H-SPNFITK—NH₂; H-SPNFITKC—NH₂; and H-SPNFIWKC—NH₂ 36.

37. The pharmaceutical composition according to claim 30, wherein the inhibitor is a heptapeptide comprising an amino acid sequence selected from the group consisting of: a) Ser-Pro-Thr-Phe-Ile-Trp-Lys; b) Ser-Pro-Thr-Phe-Thr-Trp-Lys; c) His-Pro-Thr-Phe-Thr-Trp-Lys; and d) Ser-Pro-Asn-Phe-Ile-Trp-Lys.

38. The pharmaceutical composition according to claim 36, wherein the inhibitor is a hexapeptide comprising the amino acid sequence Ser-Pro-Thr-Phe-Ile-Trp.

39. The pharmaceutical composition according to claim 29, wherein the inhibitor is capable of associating with Guanylyl Cyclase C, and said association results in cessation the catalyses of synthesis of cGMP by Guanylyl Cyclase C.

40. The pharmaceutical composition according to claim 39, wherein the inhibitor is an antagonist of Guanylin.

41. The pharmaceutical composition according to claim 40, wherein the association of the inhibitor with Guanylyl Cyclase C results in a conformational change of Guanylyl Cyclase C.

42. The pharmaceutical composition according to claim 29-41, wherein the inhibitor upon association with Guanylyl Cyclase C sterically inhibits association of Guanylyl Cyclase C with Guanylin.

43. The pharmaceutical composition according to claim 42, wherein the inhibitor is an inactive Guanylin topoisomer.

44. The pharmaceutical composition according to claim 43, wherein the inhibitor is the naturally occuring inactive Guanylin topoisomer.

45. The pharmaceutical composition according to claims 29-49, wherein the inhibitor is a polypeptide of the sequence:
H-PNTCEICA₈A₉A₁₀A₁₁CTGC—NH₂
wherein A₈, A₉, A₁₀ and A₁₁ could be any amino acid.

46. The pharmaceutical composition according to claim 45, wherein A₈ is an aliphatic amino acid.

47. The pharmaceutical composition according to claim 45, wherein A₈ is selected from the group consisting of Gly, Ala, Leu, Ile and Val.

48. The pharmaceutical composition according to claim 45, wherein A₉ is selected from the group consisting of Asn and Gln.

49. The pharmaceutical composition according to claim 45, wherein A₉ is an aromatic amino acid.

50. The pharmaceutical composition according to claim 49, wherein A₉ is substitued with halogen.

51. The pharmaceutical composition according to claim 45, wherein A₁₀ an aliphatic amino acid.

52. The pharmaceutical composition according to claim 45, wherein A₁₀ is selected from the group consisting of Gly, Ala, Leu, Ile and Val.

53. The pharmaceutical composition according to claim 50, wherein A₁₁ is an aliphatic aminoacid.

54. The pharmaceutical composition according to claim 50, wherein A₁₁ is selected from the group consisting of Gly and Ala.

55. The pharmaceutical composition according to claims 29-49, wherein the inhibitor is a polypeptide of the sequence:
H-PNTCA₅A₆CA₈A₉A₁₀A₁₁CA₁₃A₁₄C—NH₂
wherein A₅, A₆, A₈, A₉, A₁₀, A₁₁, A₁₃ and A₁₄ could be any amino acid.

56. The pharmaceutical composition according to claim 55, wherein A₅ is not Glu.

57. The pharmaceutical composition according to claim 55, wherein A₆ is not Ile.

58. The pharmaceutical composition according to claim 55, wherein A₈ is not Ala.

59. The pharmaceutical composition according to claim 55, wherein A₉ is not Tyr.

60. The pharmaceutical composition according to claim 55, wherein A₁₀ is not Ala.

61. The pharmaceutical composition according to claim 55, wherein A₁₁ is not Ala.

62. The pharmaceutical composition according to claim 55, wherein A₁₃ is not Thr.

63. The pharmaceutical composition according to claim 55, wherein A₁₄ is not Gly.

64. The pharmaceutical composition according to claim 55, wherein A₅, A₆, A₈, A₉, A₁₀, A₁₁, A₁₃ and A₁₄ are all Ala.

65. The pharmaceutical composition according to claim wherein the inhibitor is the cyclic polypeptide as defined in any of the claims 1-17.

66. The pharmaceutical composition according to claim wherein the inhibitor comprises an antibody or a fragment of an antibody.

67. The pharmaceutical composition according to claim wherein the inhibitor comprises an antibody or a fragment of an antibody, and said antibody is a monoclonal antibody.

68. The pharmaceutical composition according to claim wherein the antibody or the fragment of an antibody specifically binds to a Guanylyl Cyclase C polypeptide.

69. The pharmaceutical composition according to claim wherein the antibody or the fragment of an antibody specifically binds to a polypeptide from the extra cellular domain of Guanylyl Cyclase C.

70. The pharmaceutical composition according to claim wherein the antibody or the fragment of an antibody specifically binds to the ligand binding domain of Guanylyl Cyclase C.

71. The pharmaceutical composition according to claim wherein the inhibitor comprises the antibody according to any of the claims 18-28.

72. The pharmaceutical composition according to claim wherein the inhibitor comprises a fragment of the antibody as defined in any of the claims 18-28.

73. A compound comprising an inhibitor, wherein said inhibitor is capable of inhibiting binding of Guanylin or a compound which comprises Guanylin like activity to Guanylyl Cyclase C, the inhibitor is a polypeptide of the sequence:
H-PNTCEICA₈A₉A₁₀A₁₁CTGC—NH₂
wherein A₈, A₉, A₁₀ and A₁₁ could be any amino acid.

74. The compound according to claim 73, wherein A₈ is an aliphatic amino acid.

75. The compound according to claim 74, wherein A₈ is selected from the group consisting of Gly, Ala, Leu, Ile and Val.

76. The compound according to claim 74, wherein A₉ is selected from the group consisting of Asn and Gln.

77. The compound according to claim 74, wherein A₉ is an aromatic amino acid.

78. The compound according to claim 77, wherein A₉ is substitued with halogen.

79. The compound according to claim 74, wherein A₁₀ an aliphatic amino acid.

80. The compound according to claim 74, wherein A₁₀ is selected from the group consisting of Gly, Ala, Leu, Ile and Val.

81. The compound according to claim 74, wherein A₁₁ is an aliphatic amino acid.

82. The compound according to claim 74, wherein A₁₁ is selected from the group consisting of Gly and Ala.

83. A compound comprising an inhibitor, wherein said inhibitor is capable of inhibiting binding of Guanylin or a compound which comprises Guanylin like activity to Guanylyl Cyclase C, the inhibitor is a polypeptide of the sequence:
H-PNTCA₅A₆CA₈A₉A₁₀A₁₁CA₁₃A₁₄C—NH₂
wherein A₅, A₆, A₈, A₉, A₁₀, A₁₁, A₁₃ and A₁₄ could be any amino acid.

84. The compound according to claim 83, wherein A₅ is not Glu.

85. The compound according to claim 83, wherein A₆ is not Ile.

86. The compound according to claim 83, wherein A₈ is not Ala.

87. The compound according to claim 83, wherein A₉ is not Tyr.

88. The compound according to claim 83, wherein A₁₀ is not Ala.

89. The compound according to claim 83, wherein A₁₁ is not Ala.

90. The compound according to claim 83, wherein A₁₃ is not Thr.

91. The compound according to claim 83, wherein A₁₄ is not Gly.

92. The compound according to claim 83, wherein A₅, A₆, A₈, A₉, A₁₀, A₁₁, A₁₃ and A₁₄ are all Ala.

93. Use of a cyclic polypeptide as defined in any of the claims 1-17 for preparation of a medicament.

94. Use of an antibody polypeptide as defined in any of the claims 18-28 for preparation of a medicament.

95. Use of a compound as defined in any of the claims 82-92 for preparation of a medicament.

96. The use according to any of the claims 94-95, for preparation of a medicament for treatment of one or more symptoms of Ménière's disease.

97. The use according to claim 96, wherein said symptom is selected from the group consisting of: fluctuating hearing levels, sensation of fullness in the ear, roaring tinnitus and episodic vertigo.

98. The use according to claim 96, wherein said treatment is ameliorating.

99. The use according to claim 96, wherein said treatment is curative.

100. The use according to claim 96, wherein said treatment is prophylactic.

101. A method of treatment of one or more symptoms of Ménière's disease, comprising administrating to an individual in need thereof the pharmaceutical composition according to any of the claims 29-73 and/or a cyclic polypeptide as defined in any of claims 1-17, an antibody as defined in any of the claims 18-28 or a compound as defined in any of the claims 74-93.

102. The method according to claim 101, wherein said symptom is selected from the group consisting of: fluctuating hearing levels, sensation of fullness in the ear, roaring tinnitus and episodic vertigo.

103. The method according to claim 107, wherein said treatment is ameliorating.

104. The method according to claim 107, wherein said treatment is curative.

105. The method according to claim 107, wherein said treatment is prophylactic.

106. The method according to claim 107, wherein said individual is a human being.

107. The method according to claim 107, wherein said administration is parenteral.

108. The method according to claim 107, wherein said administration is oral.

109. A method of making a stabilised guanylin analogue comprising making a guanylin,
and adding at least one charged amino acid to the C-terminus of the guanylin, thereby making the stabilised guanylin analogue, or comprising making the satilised guanylin analogue using a standard solid phase peptide synthesis method as described herein.

110. The method of claim 109, wherein between from about 1 to about 25 charged amino acids are added to the guanylin.

111. The method of claim 110 wherein the charged amino acids are selected from the group of amino acids having propensity factor Pα> about 0.57 and a propensity factor Pβ less than or equal to about 1.10.

112. The method of claim 111, wherein at least one of the charged amino acids is lysine, arginine, aspartic acid, or glutamic acid.

113. The method of claim 112, wherein the charged amino acids added to the guanylin are the same.

114. The method of claim 113, wherein the following amino acid sequence is added to the guanylin C-terminus to stabilise the peptide: Asp, Asp-Asp, Asp-Asp-Asp, Lys, Asp-Lys, Asp-Lys-Lys, Asp-Lys-Lys-Lys, Asp-Lys-Lys-Lys-Lys, Asp-Lys-Lys-Lys-Lys-Lys, Asp-Lys-Lys-Lys-Lys-Lys-Lys, Lys-Lys, Lys-Lys-Lys, Lys-Lys-Lys-Lys-, Lys-Lys-Lys-Lys-Lys- or Lys-Lys-Lys-Lys-Lys-Lys.

115. The guanylin analogues selected from the group consisting of mouse guanylin—K₆—OH/NH₂, mouse guanylin-D—K₆—OH/NH₂, mouse guanylin-D—K—OH/NH₂, mouse guanylin-D—OH/NH₂, mouse desP¹,N²-guanylin-L—K₂—OH/NH₂, mouse desP¹, N²-guanylin-L—OH/NH₂, mouse desP¹,N²-guanylin-D—K₆—OH/NH₂, mouse desP¹,N²-guanylin-D—OH/NH₂, and mouse desP¹,N²-guanylin—OH/NH₂.

116. Use of the guanylin analogues of claim 115 for the preparation of a medicament for the treatment of colorectal cancer.

117. Use according to claim 116 wherein said cancer is colonic carcinoma.

118. A method for screening compounds for capacity to modulate a membrane bound guanylyl cyclase signaling receptor (GC-C) comprising:
a) contacting cells expressing the GC-C with the compound under conditions conducive to forming cGMP; and
b) measuring the cGMP produced by the cells, wherein a change in cGMP production relative to a control (vehicle) is taken to be indicative of the compound that modulates the GC-C.

119. The method of claim 118, wherein the compound exhibits an IC50 of at least 100 μM in the assay.

120. The method of claim 118 or 119, wherein the compound exhibits an EC50 of at least 1 μM in the assay.

121. A method for screening compounds for capacity to modulate a membrane bound guanylyl cyclase signaling receptor (GC-C) in an animal comprising:
a) administering a candidate compound to the animal under conditions suitable for detecting diuresis associated with a sustained natriuretic response that outlasts the diuretic response, and
b) measuring the diuresis and urinary sodium excretion produced by the animal, wherein presence of diuresis relative to a control (vehicle) is taken to be indicative of capacity to modulate the GC-C in the animal.

122. The method of claim 122, wherein the compound exhibits an increase in diuresis of at least about 20% when compared to a control.

123. The method of claim 118, wherein the method further comprises:
c) measuring at least one of diuresis, urinary sodium excretion and urinary potassium excretion produced by the animal, wherein presence of the diuresis, urinary sodium excretion and urinary potassium excretion relative to a control (vehicle) is taken to be indicative of capacity to modulate the GC-C in the animal.

124. A method for screening compounds for capacity to modulate a membrane bound guanylyl cyclase signaling receptor (GC-C) in an animal comprising:
a) administering a GC-C receptor agonist to the animal under conditions suitable for detecting otoacoustic emission;
b) administering a candidate compound exhibiting GC-C receptor antagonist or inhibitor activity to the animal under conditions suitable for detecting otoacoustic emission; and
c) measuring the otoacoustic emission, wherein the emission having the same magnitude relative to a control (vehicle) is taken to be indicative of capacity to antagonise the effect of the GC-C agonist in the animal.

125. The method of claim 124, wherein the compound exhibits a change in otoacoustic emission of at least about 5 dB.

126. The method of claims 118, 121, or 123, wherein the method further comprises testing identified compounds by administering a GC-C receptor agonist to the animal under conditions suitable for detecting otoacoustic emission;
administering a candidate compound exhibiting GC-C receptor antagonist or inhibitor activity to the animal under conditions suitable for detecting otoacoustic emission measuring otoacoustic emission in the animal, wherein the emission having the same magnitude relative to a control (vehicle) is taken to be indicative of capacity to antagonise the effect of the GC-C agonist in the animal.

127. A method for facilitating diuresis in a mammal, the method comprising administering a therapeutically effective amount of at least one of: guanylin or a biologically active fragment thereof; guanylin analogue or cyclic peptide.

128. Use of a peptide sequence of or an epitopic fragment of the GC-C receptor preferably coupled to a carrier through a terminal cysteinyl residue for raising antibodies capable of specifically binding to said peptide.

129. Use of Compound 11 H-SPNFITKC—NH₂ (Compound 11) or H-SPNFIWKC—NH₂ (Compound 11A) for raising an antibody capable of specifically binding to a GC-C receptor or a fraction of the peptide sequence thereof.

130. The antibody of claim 129, wherein the antibody has an IgG serotype.

131. The antibody of claim 128 or 129, wherein the antibody is polyclonal or monoclonal.

132. The antibody of claim 131, wherein the antibody is polyclonal and specifically binds one of the following peptides: H-SPNFIWKC—NH₂, and H-SPNFITKC—NH₂ (Compound 11).

133. A specific IgG antibody against the GC-C receptor produced as described herein.

134. A method for preventing or treating hypotension, the method comprising administering a therapeutically effective amount of a compound disclosed herein; and preventing or treating the hypotension.

135. A method of preventing or treating erectile dysfunction, the method comprising administering a therapeutically effective amount of a compound disclosed herein; and preventing or treating the hypotension.
